SEQUENCE LISTING (1 sequence)

SEQ ID NO: 1 - protein, 39 aa, Artificial Sequence
  insulin analogue/derivative
HGEGTFTSDLSKQMEEEAVRLFIEWLKNGGPSSGAPPPS

(19) United States
(12) Patent Application Publication
(10) Pub. No.: US 2016/0296710 A1
(43) Pub. Date: Oct. 13, 2016

(54) DRIVE ASSEMBLY FOR A DRUG DELIVERY DEVICE AND DRUG DELIVERY DEVICE COMPRISING A DRIVE ASSEMBLY

(71) Applicant: SANOFI, Paris (FR)

(72) Inventors: Michael Bainton, Kineton, Warwickshire (GB); William Marsh, Buckingham, Buckinghamshire (GB); Matthew Jones, Warwick, Warwickshire (GB); Simon Lewis Bilton, Warwickshire (GB)

(21) Appl. No.: 14/914,866

(22) PCT Filed: Sep. 3, 2014
(86) PCT No.: PCT/EP2014/068648

§ 371 Date: Feb. 26, 2016

(30) Foreign Application Priority Data
Sep. 3, 2013 (EP) 13182753.7

(51) Int. Cl. A61M 5/315; A61M 5/24; A61M 5/20

(52) U.S. Cl. CPC A61M 5/31568; A61M 5/31536; A61M 5/31583; A61M 5/31541; A61M 5/2033; A61M 5/31593; A61M 5/24; A61M 5/31553; A61M 5/31563; A61M 2005/3126

(57) ABSTRACT

A drive assembly (180, 201, 301) for a drug delivery device (101, 354) is provided, the drive assembly (180, 201, 301) comprising a dose setting member (122, 203, 303) for setting a dose of a drug, an indicator (128, 243, 343) for indicating the size of the set dose, a piston rod (102, 214, 314) and a dispense stop for limiting a movement of the piston rod (102, 214, 314) and/or a movement of the indicator (128, 243, 343) during dose dispense, wherein the dispense stop comprises a stop feature (170, 233, 333) configured to move during dose setting.

The present disclosure relates to a drive assembly for a drug delivery device and a drug delivery device comprising a drive assembly. In particular, the drug delivery device may be configured to deliver variable user selectable doses of a medicinal product. The drug delivery device may be spring driven. As an example, the drug delivery device may be an autoinjector, in particular a semi-automatic autoinjector.

It is an object of the present invention to disclose a drive assembly having improved properties.

According to a first aspect, a drive assembly for a drug delivery device is provided. The drive assembly comprises a dose setting member for setting a dose of a drug and an indicator for indicating the size of the set dose.

As an example, the dose setting member may be rotated for setting a dose of a drug. The amount of rotation of the dose setting member may determine the size of the set dose. In particular, a user may vary the size of the set dose by operating the dose setting member. The indicator may comprise numbers and/or graduations for indicating the size of the set dose. During a dose setting operation, the indicator may move such that a number or graduation corresponding to the currently set dose is displayed to a user.

The drive assembly may comprise a spring member. The spring member may provide a force for dispensing the set dose. In particular, the drive assembly may comprise a piston rod, wherein the spring member is configured to drive the piston rod towards a dispensing end of a drug delivery device during a dose dispense operation. The spring member may be a compression spring or a torsion spring, for example. The spring member may be tensioned during a dose setting operation. Alternatively, the spring member may be configured such that a tensioning before the first use of the device is sufficient to deliver all doses. In this case, a further tensioning during dose setting may not be required. The spring member may relax during a dose dispensing operation.

According to an embodiment, the indicator of the drive assembly may be configured to move towards an initial position during dose dispense. An initial position of the indicator may be a position in which the indicator indicates a zero size of a dose. Accordingly, the indicator indicates that no dose is set.

The drive assembly may comprise a piston rod. As an example, the piston rod may be configured as a lead screw or a toothed rack. The piston rod may be rigid or flexible. The piston rod may be configured to act on a piston in a cartridge, in particular to move the piston during a dose dispense operation. The piston rod may comprise or may be connected to a bearing, wherein the piston rod may act on the piston via the bearing.

During dose dispensing, the indicator may be coupled to the piston rod. The indicator may be coupled to the piston rod via a coupling member. In particular, “coupling” may mean that a movement of the piston rod results in a movement of the indicator. Due to the coupling, the indicator may be moved to its initial position when the piston rod moves in a dose dispense operation. Thereby, the indicator is automatically reset to its initial position when the set dose has been dispensed.

In one embodiment, an operation of the dose setting member may cause a decoupling of the indicator from the piston rod. In particular, the indicator may be releasably coupled to a reversing member coupled to the piston rod. The indicator may be decoupled from the reversing member by an operation of the dose setting member. As an example, the drive assembly may be configured such that the dose setting member has to be depressed before a dose can be set. A depression of the dose setting member may cause the decoupling. In particular, the depression may move the coupling member, which may be permanently connected to the indicator, out of engagement with the reversing member. The reversing member may be coupled to the piston rod, in particular permanently coupled to the piston rod. The coupling may be directly or via a further member, for example a drive control member which controls the movement of the piston rod. After the operation of the dose setting member, the indicator may be re-coupled to the piston rod, in particular re-coupled to the reversing member.

In a further embodiment, the drive assembly may comprise an actuator for initiating a dispensing of a dose, in particular after a dose has been set. In particular, the indicator may be coupled to a reversing member coupled to the piston rod. An operation of the actuator may cause a coupling of the indicator to the piston rod. As an example, the actuator may result in an engagement of a coupling member, which may be connected to the indicator, with the reversing member. The reversing member may be coupled to the piston rod, in particular engaged with the piston rod, and may be driven by the piston rod during a dose dispense operation. After the operation of the actuator member, the indicator may be decoupled from the piston rod, in particular decoupled from the reversing member.

Furthermore, during dose setting the indicator may be coupled to the dose setting member. Thereby, a movement of the dose setting member may result in a movement of the indicator. The indicator may be permanently connected to the dose setting member. Alternatively, during dose dispensing the indicator may be decoupled from the dose setting member. As an example, an operation of an actuator may cause the decoupling.

According to an embodiment, the drive assembly comprises a dispense stop for limiting a movement of a piston rod and/or a movement of the indicator during dose dispense. By limiting the movement of the piston rod, the dispense stop may ensure that the correct amount of the dose, i.e. the amount of the set dose, is dispensed. By limiting the movement of the indicator, the dispense stop may ensure that the indicator returns to its initial position.

The dispense stop may comprise a stop feature. The stop feature may be configured to move, in particular rotate, during dose setting. Thereby, an end stop position of the stop feature may be set. The end stop position of the stop feature may define the end stop position of the indicator and/or the piston rod. The stop feature may be connected to the indicator, in particular permanently connected to the indicator. As an example, the stop feature may be an integral part of the indicator. Alternatively, the stop feature may be coupled to the indicator during dose setting. As an example, the stop feature may be connected to a further member, which may be coupled to the indicator during dose setting. The further member may be decoupled from the indicator during dose dispensing.

The stop feature may be fixed during dose dispensing. In particular, the stop feature may be fixed relative to a housing of the drive assembly.

The drive assembly may comprise a further stop feature. The movement of the piston rod and/or the indicator may be stopped by an abutment of the stop feature and the further stop feature. The further stop feature may be connected to, in particular be an integral part of, a member coupled to the piston rod during dose dispense. As an example, the further stop feature may be connected to a drive control member, which controls the movement of the piston rod. Alternatively, the further stop feature may be connected to the housing of the drive assembly, for example may be an integral part of the housing.

The further stop feature may be configured to move during dose dispensing and may be fixed during dose setting, in particular fixed to the housing. Alternatively, the further stop member may be permanently fixed to the housing. In particular, when the stop feature is enabled to move, the further stop feature may be disabled from moving and vice versa.

According to a further aspect, a drug delivery device comprising a drive assembly is provided. The drive assembly may be the drive assembly disclosed above such that every structural and functional feature disclosed with respect to that drive assembly may also be present in the drug delivery device.

The drug delivery device may further comprise a cartridge comprising a piston wherein the drive assembly is adapted to provide a force on the piston such that the piston is moved in the distal direction further into the cartridge. Thereby, a drug may be expelled from the cartridge.

The term “distal end” may describe an end of the device or a part thereof which is closest to a dispensing end of the device. The term “proximal end” may describe an end of the device or a part thereof which is furthest away from the dispensing end of the device. Analogously, the term “distal direction” may describe a direction towards a dispensing end of the device and the term “proximal direction” may describe a direction away from the dispensing end of the device.

The drug delivery device may be an injection device. The medicament may be delivered to a user by means of a needle. The drug delivery device may be configured for multiple dose applications. The drug delivery device may be a pen-type device. The drug delivery device may be disposable. The term “disposable” means, that the drug delivery device cannot be reused after an available amount of a medication has been delivered from the drug delivery device. The drug delivery device may be configured to deliver a liquid medication. The medication may be, for example, insulin.

According to an embodiment, the drive assembly comprises a last dose stop for preventing a setting of a dose larger than an available amount of the drug.

In particular, the last dose stop may prevent a further operation of the dose setting member in a dose setting direction when the available dose has been set. However, in this state, the last dose stop may allow a movement of the dose setting member in a dose cancelling direction in order to decrease the size of the set dose. The dose cancelling direction may be opposite to the dose setting direction. Furthermore, when the available dose has been set, dispensing a last dose may be enabled.

The last dose stop may comprise a last dose stop member. The last dose stop member may be configured to move towards an end position during the setting of a dose. When the last dose stop member is at the end position, a further increase of the size of the set dose may be prevented. In particular, in the end position, a movement of the last dose stop member in at least one direction may be prevented. Thereby, also a movement of the dose setting member in the dose setting direction may be prevented.

During dose setting, the last dose stop member may be coupled to the dose setting member such that an operation of the dose setting member results in a movement of the last dose stop member. As an example, the last dose stop member may be coupled to the dose setting member via further members of the drive assembly. During a movement of the dose setting member in a dose cancelling direction, for example when decreasing or fully cancelling a set dose, the last dose stop member may move towards a start position. Thereby, the available dose always corresponds to the position of the last dose stop member. During a dose dispensing operation, the last dose stop member may be decoupled from the dose setting member.

The drive assembly may comprise a housing. The last dose stop member may be rotationally fixed to the housing. In particular, the last dose stop member may be rotationally fixed to the housing both during dose setting and dose dispensing operations. A translational movement of the last dose stop member relative to the housing may be allowed.

The drive assembly may comprise a last dose stop drive member. In particular, the last dose stop member may be engaged, for example threadedly engaged, with the last dose stop drive member. The last dose stop drive member may be configured to drive the last dose stop member, in particular cause a movement of the last dose stop member during a dose setting operation and/or a dose cancelling operation.

The last dose stop drive member may be translationally fixed to a housing, in particular permanently translationally fixed to the housing. During a dose setting operation, a rotational movement of the last dose stop drive member may be enabled. In particular, the last dose stop drive member may be configured to rotate during a dose setting operation. Thereby, the last dose stop member may be moved along the last dose stop drive member. During a dose dispensing operation, the last dose stop drive member may be rotationally and translationally fixed to the housing. Thereby, any movement of the last dose stop member may be prevented.

Furthermore, a setting of a dose larger than an available amount of the drug may be prevented by an interaction of the last dose stop member with the last dose stop drive member. In particular, the last dose stop member may comprise a stop face and the last dose stop drive member may comprise a stop face. When the stop faces abut, a further setting of a dose may be prevented. In particular, the position of the stop faces during an abutment may define an end position of the last dose stop member.

The terms “medicinal product”, “medication” or “drug”, as used herein, preferably mean a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-idecanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu- Lys-Asn-Gly-Gly-Pro-Ser-Ser-Gly-Ala-Pro- Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Further features, refinements and expediencies become apparent from the following description of the exemplary embodiments in connection with the figures.

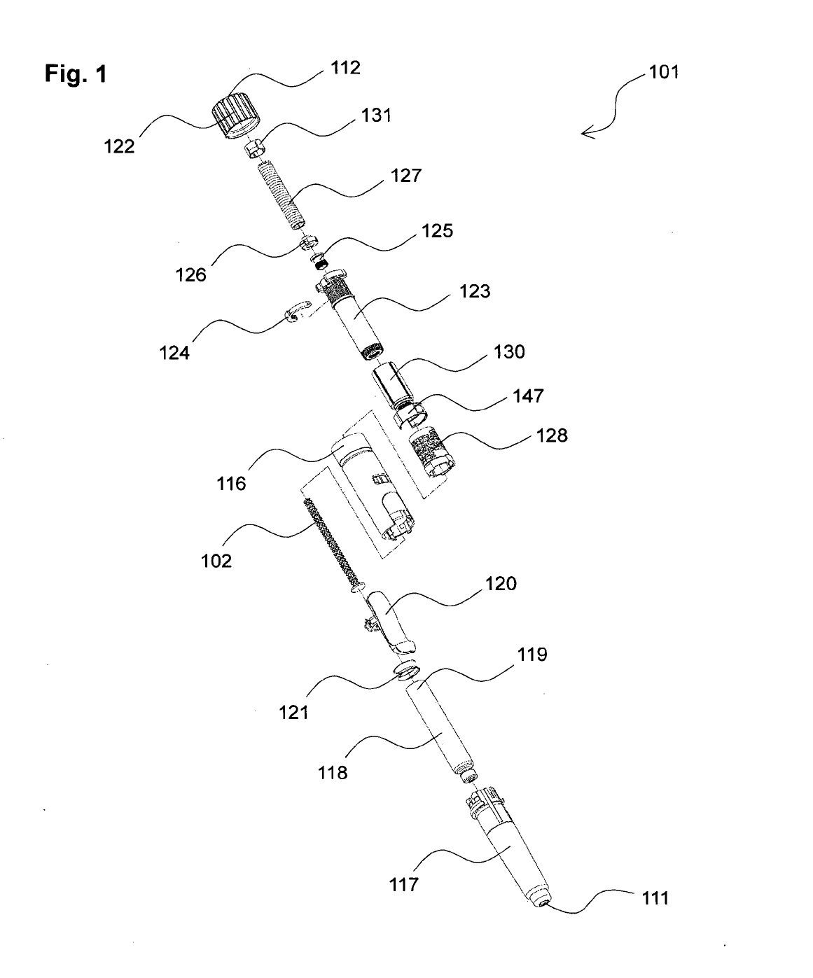

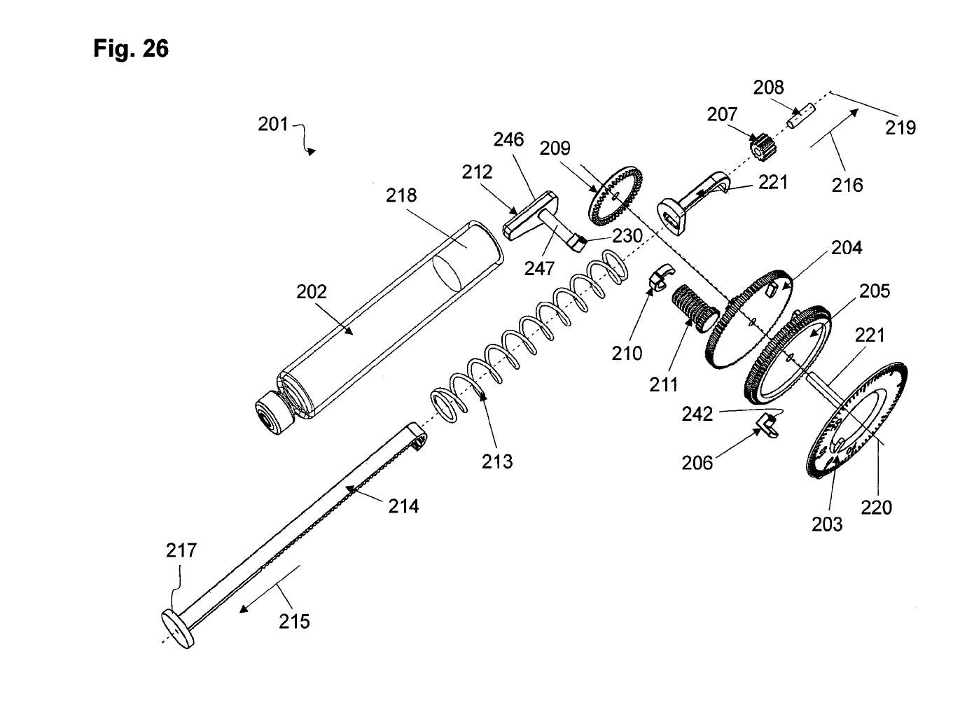

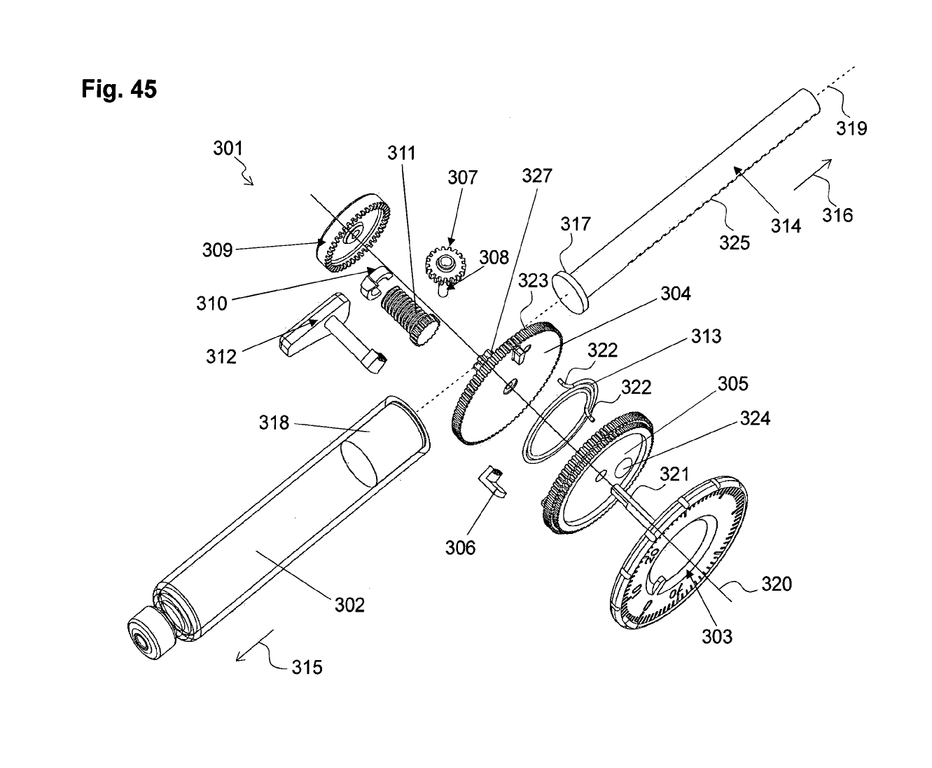

FIGS. 1 to 25C relate to a first embodiment of a drive assembly and a drug delivery device. FIGS. 26 to 44 relate to a second embodiment of a drive assembly and a drug delivery device. FIGS. 45 to 58 relate to a third embodiment of a drive assembly and a drug delivery device.

FIG. 1 shows an exploded view of a drug delivery device comprising a drive assembly according to a first embodiment,

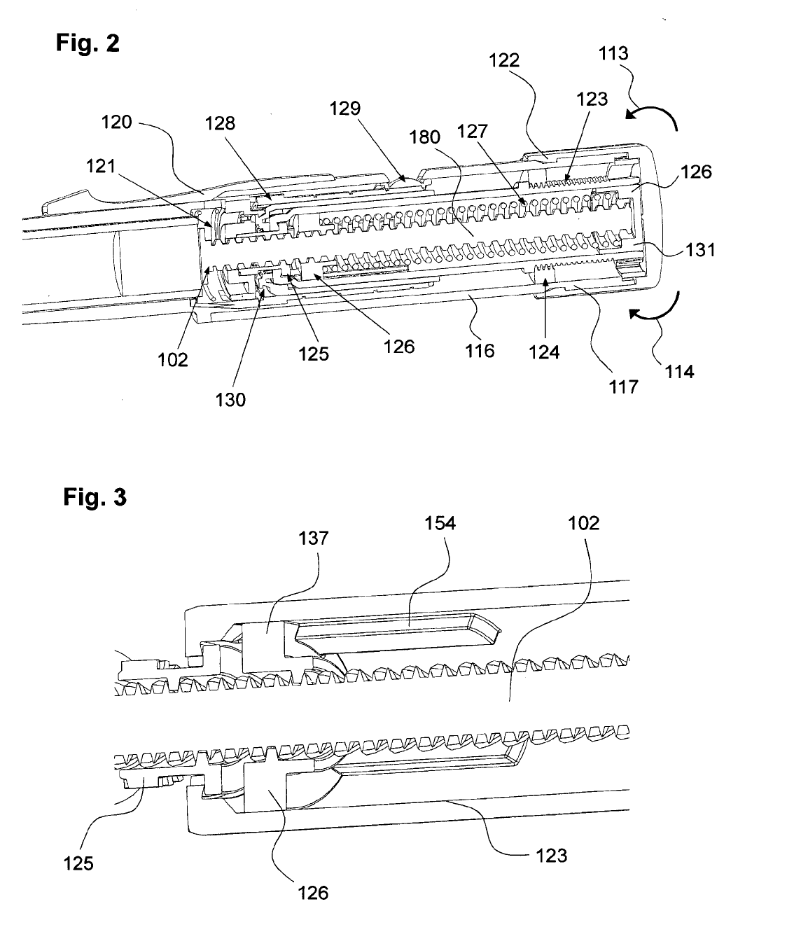

FIG. 2 shows a sectional view of the drug delivery device of FIG. 1 in an assembled state,

FIG. 3 shows a detailed view of the rotation member, the piston rod, the piston rod nut and the locking member,

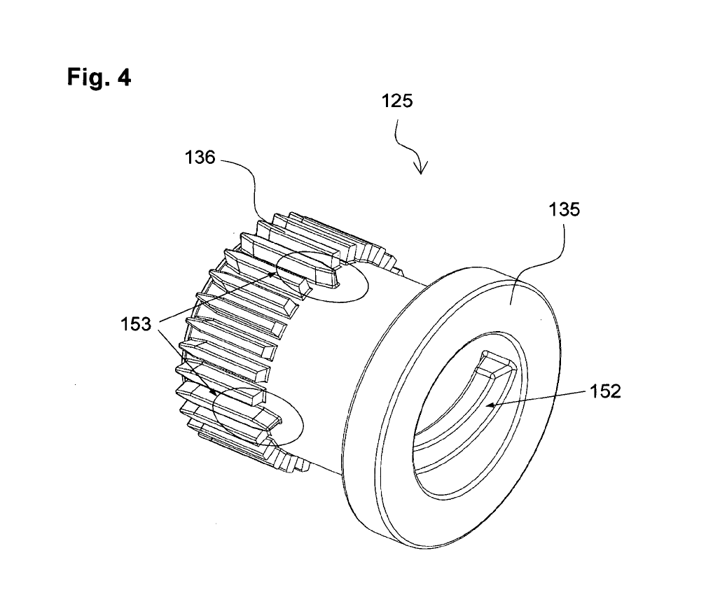

FIG. 4 shows a detailed view of the locking member,

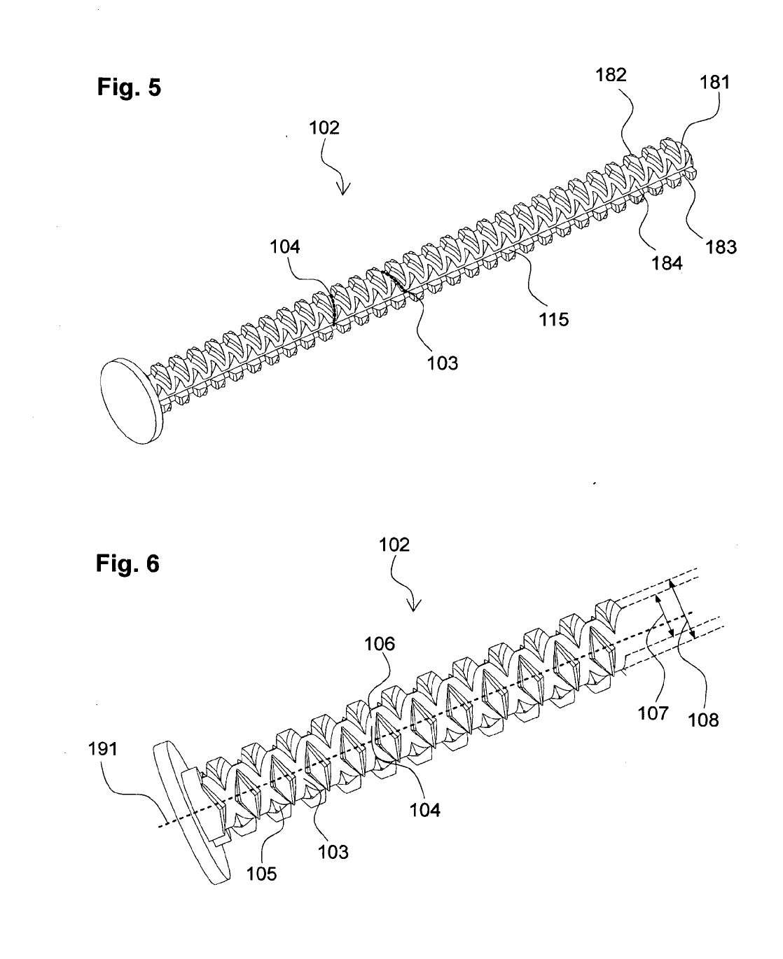

FIG. 5 shows a schematic view of the piston rod,

FIG. 6 shows a further embodiment of the piston rod,

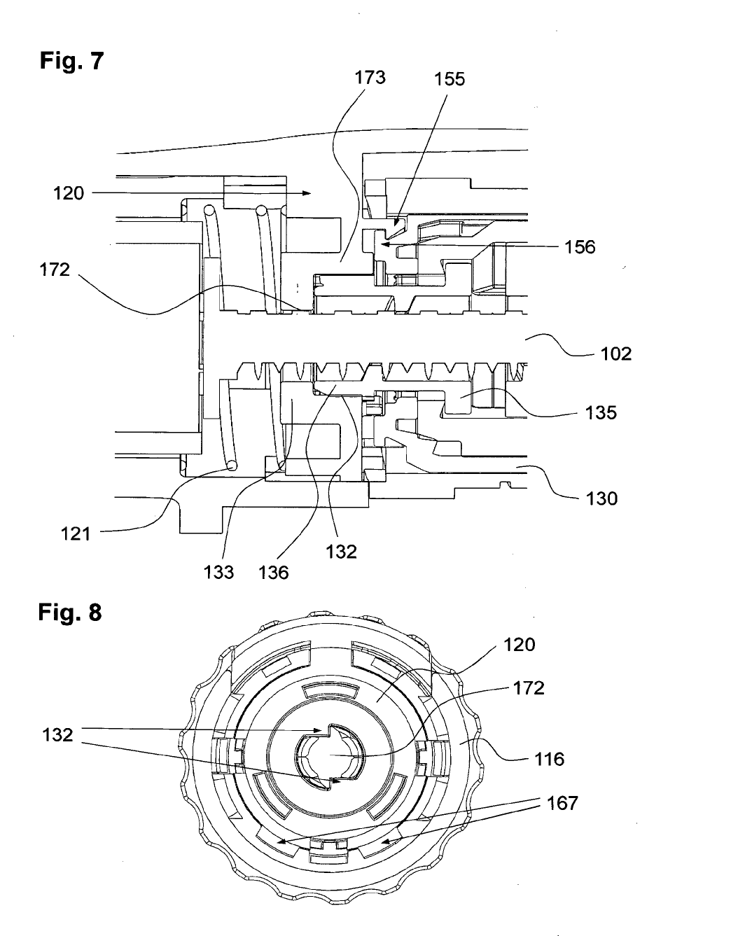

FIG. 7 shows a sectional view of the engagement of the actuator with the piston rod, the locking member and the coupling member during the setting of a dose,

FIG. 8 shows the assembly of FIG. 7 in a different sectional view,

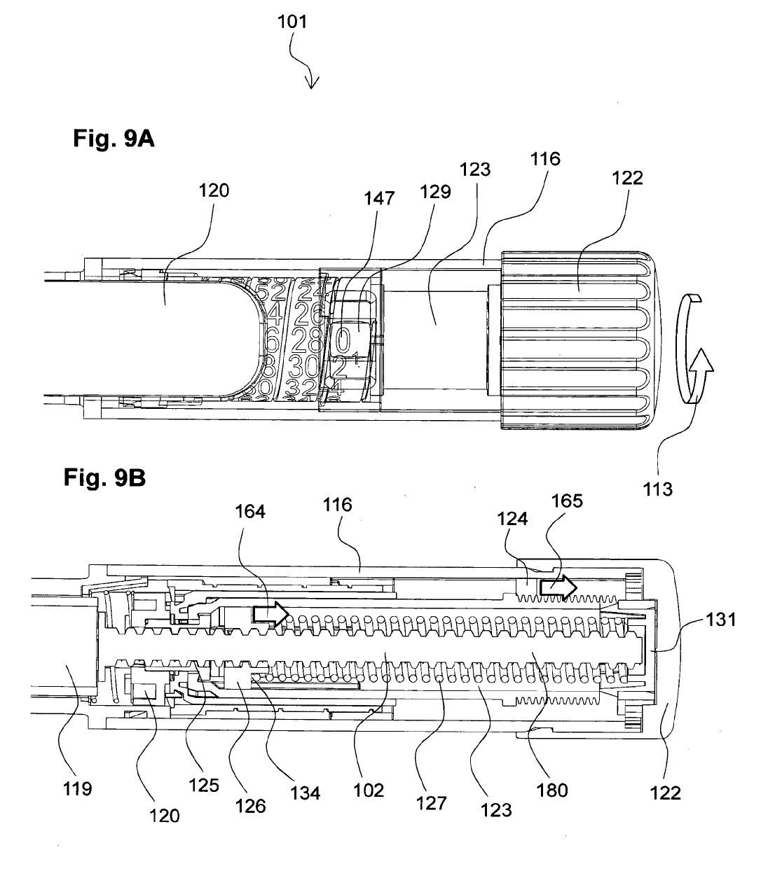

FIGS. 9A and 9B show sectional views of a proximal part of the drug delivery device in an assembled state,

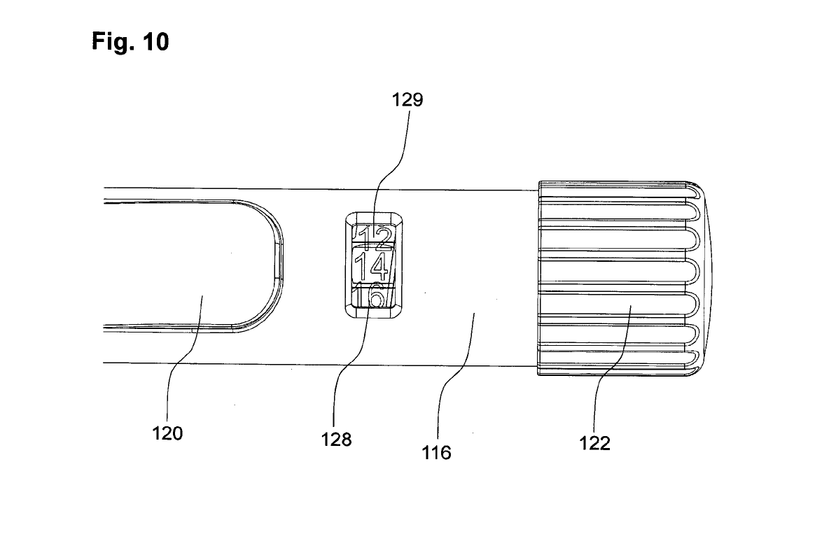

FIG. 10 shows the proximal part of a drug delivery device with an amount of a set dose being displayed in an indication window,

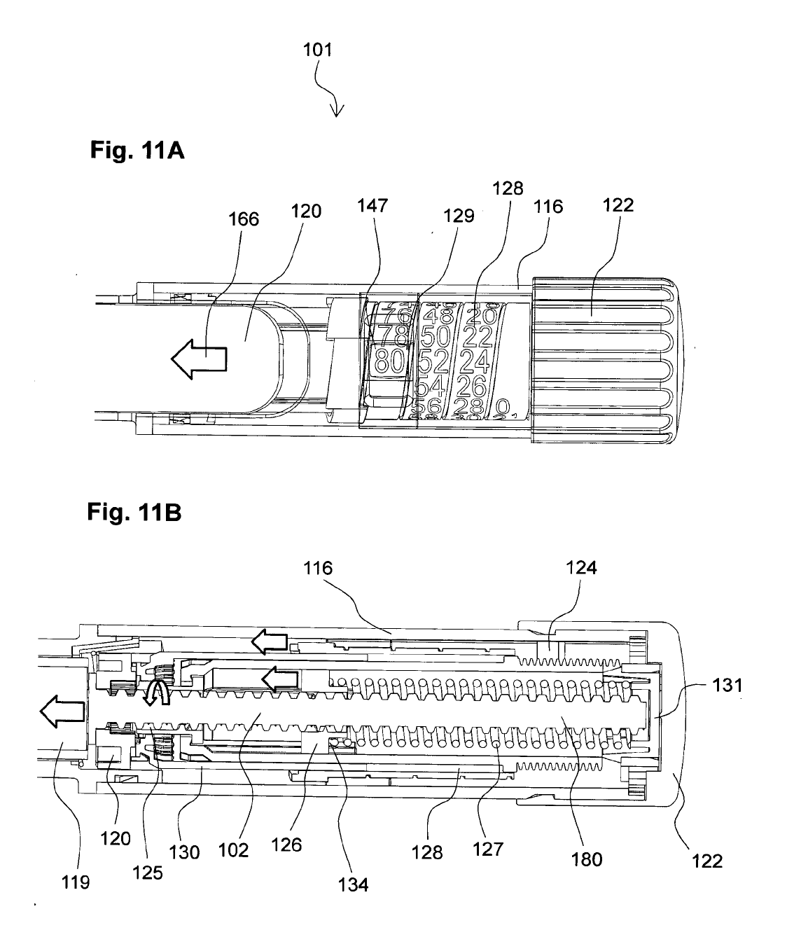

FIGS. 11A and 11B show sectional views of the drug delivery device of FIGS. 9A and 9B in a state where a dose has been set,

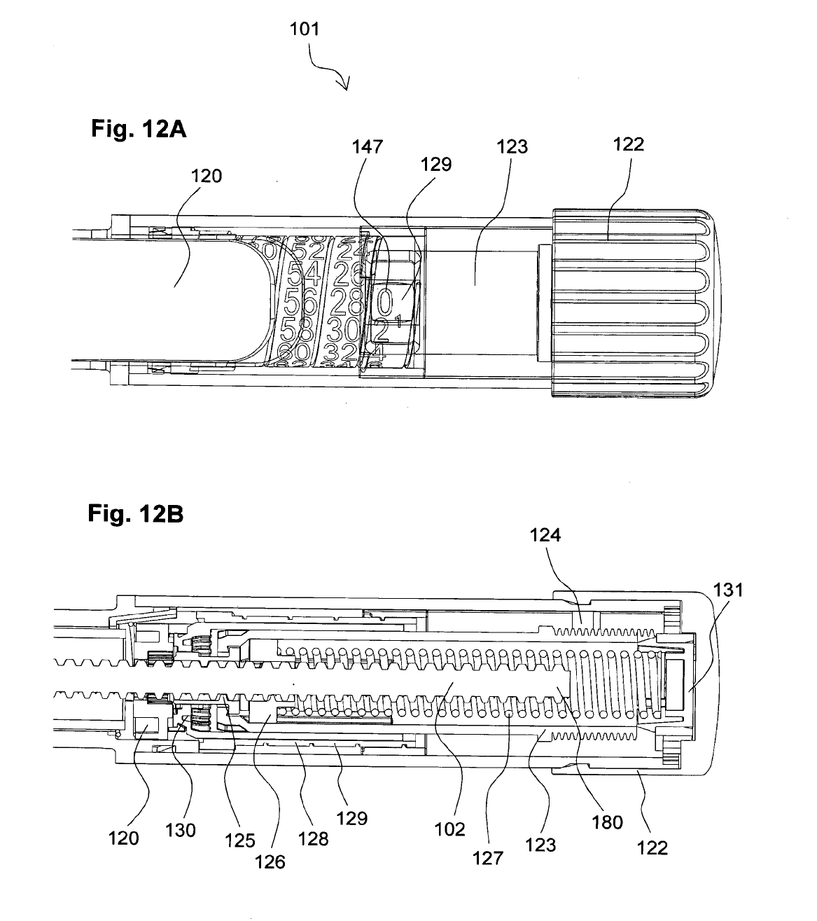

FIGS. 12A and 12B show sectional views of the drug delivery device of FIGS. 9A, 9B, 11A and 11B in a condition when a dose of medication has been delivered from the device,

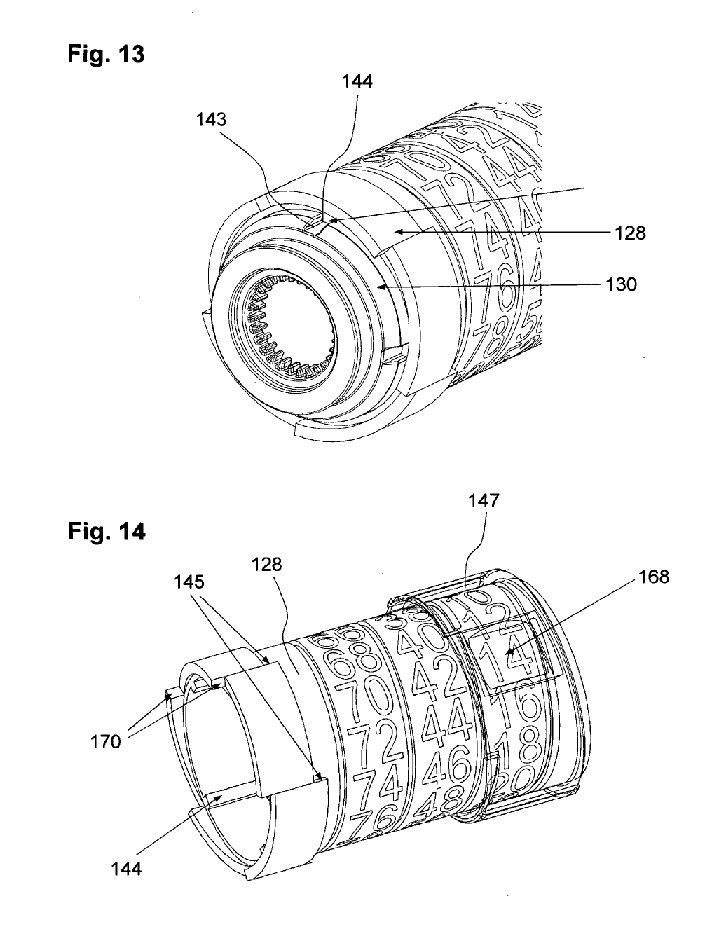

FIG. 13 shows the coupling member engaged with an indicator in a detailed view,

FIG. 14 shows a schematic view of the indicator and a window member,

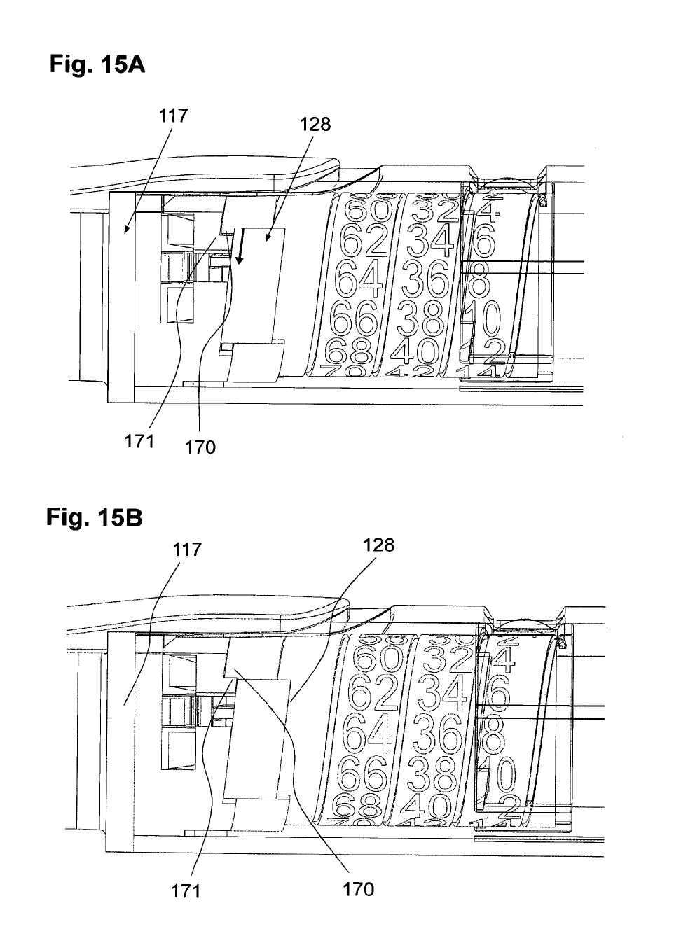

FIGS. 15A and 15B show a partial section of a cartridge holder and the indicator,

FIG. 16 shows a part of the window member,

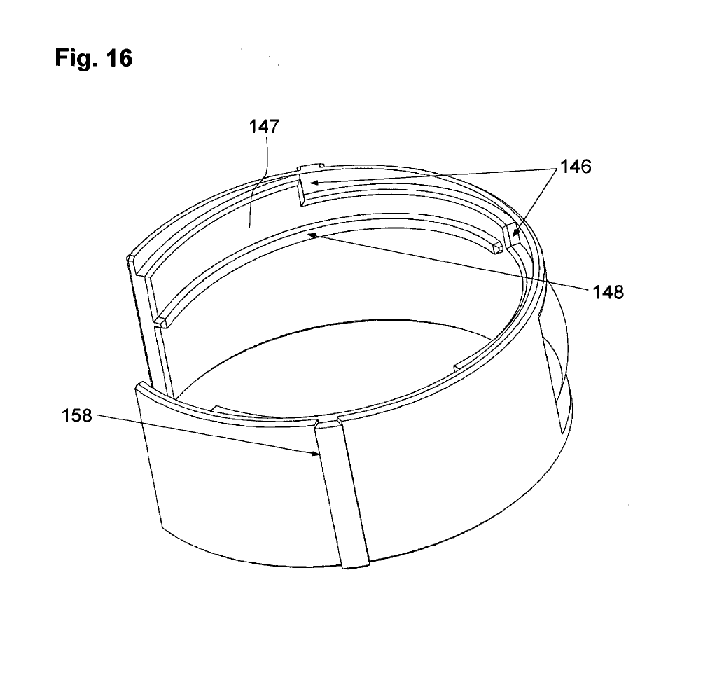

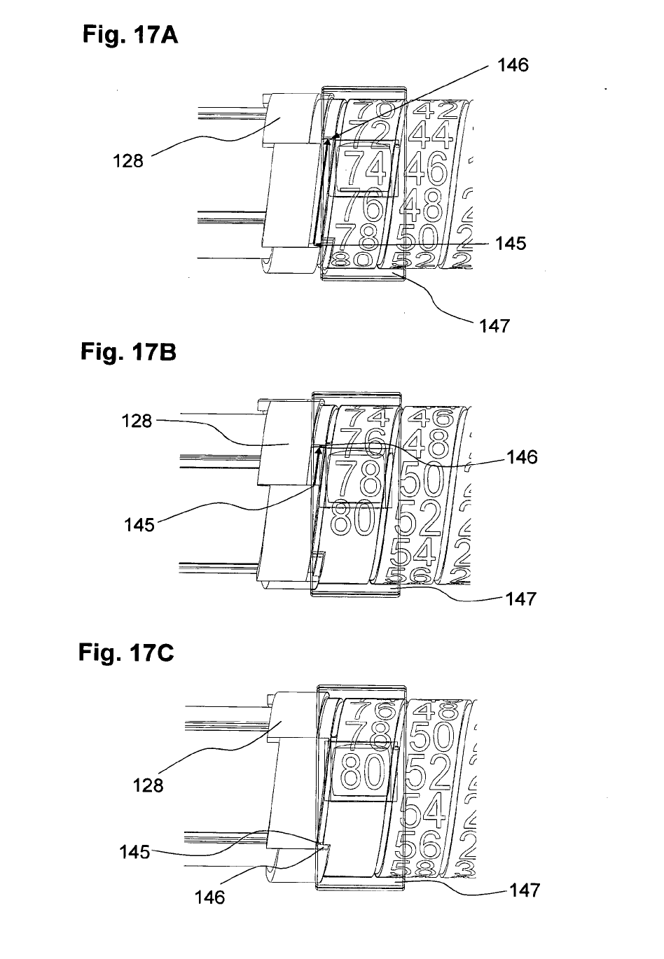

FIGS. 17A to 17C show the window member and the indicator in three different states during an operation of the drug delivery device,

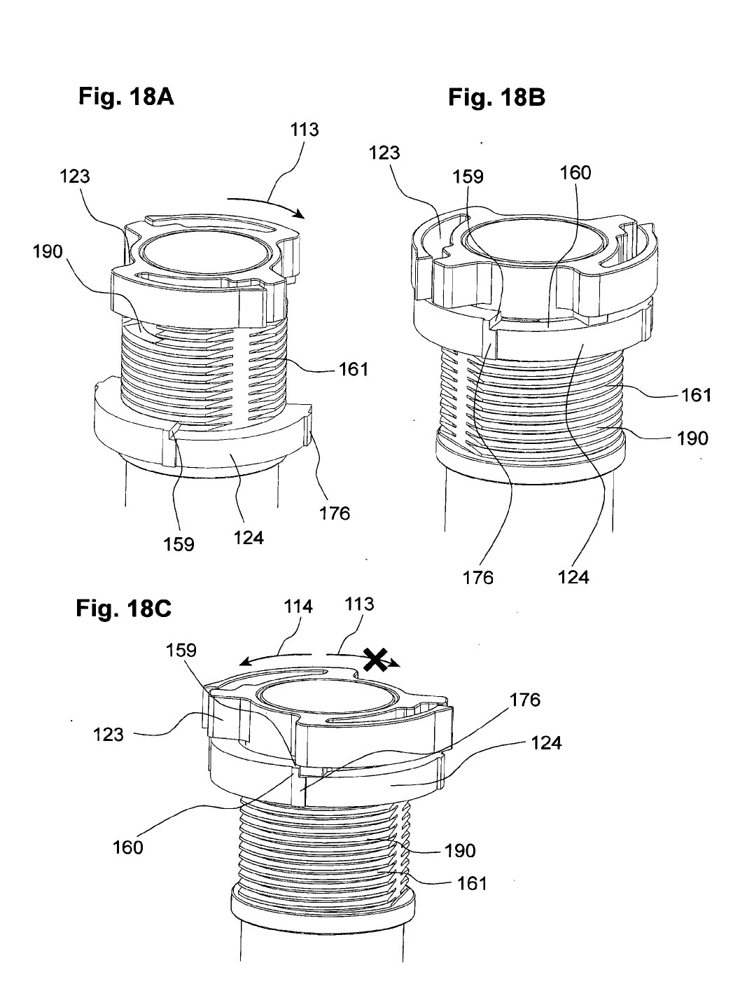

FIGS. 18A to 18C show the engagement of the last dose stop member with the rotation member in three different states,

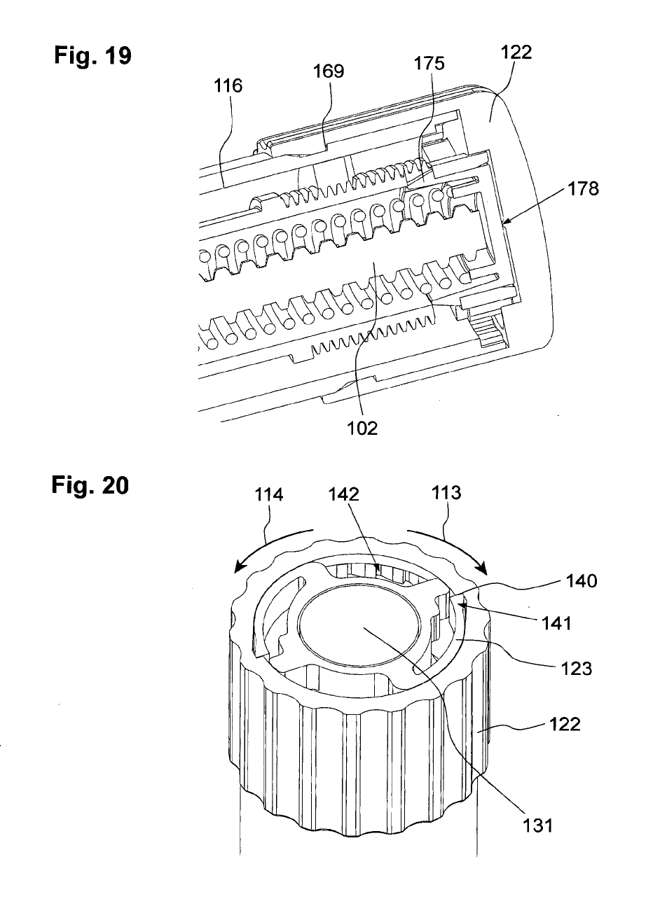

FIG. 19 shows a section through the proximal end of the drug delivery device,

FIG. 20 shows a sectional view of the engagement of the rotation member with the dose setting member,

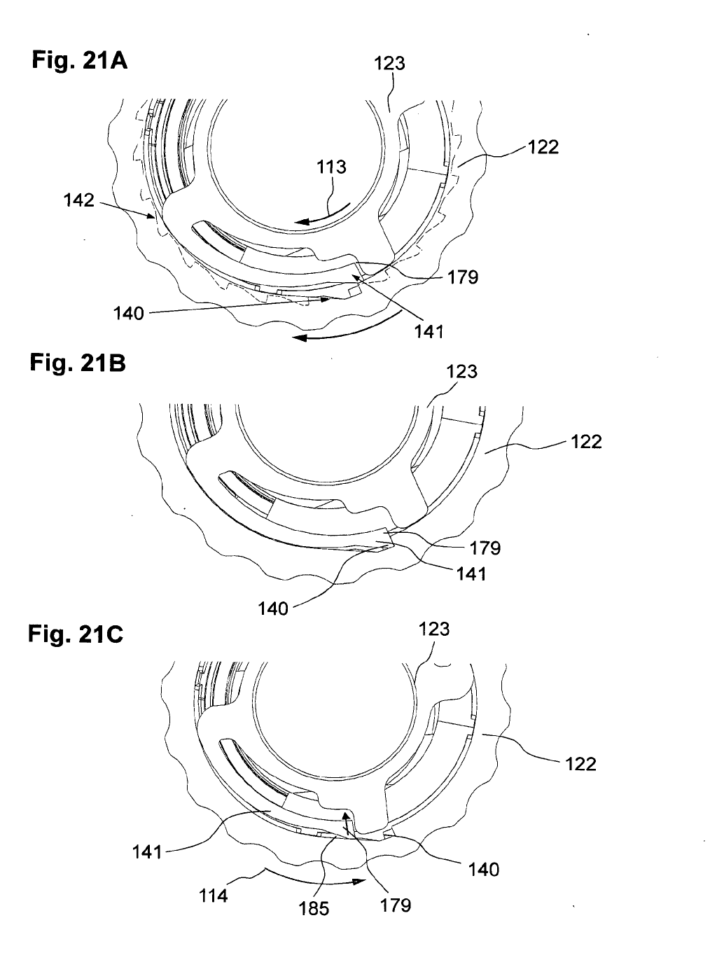

FIGS. 21A to 21C show sectional views of the dose setting member and the rotation member according to FIG. 20 in three different states,

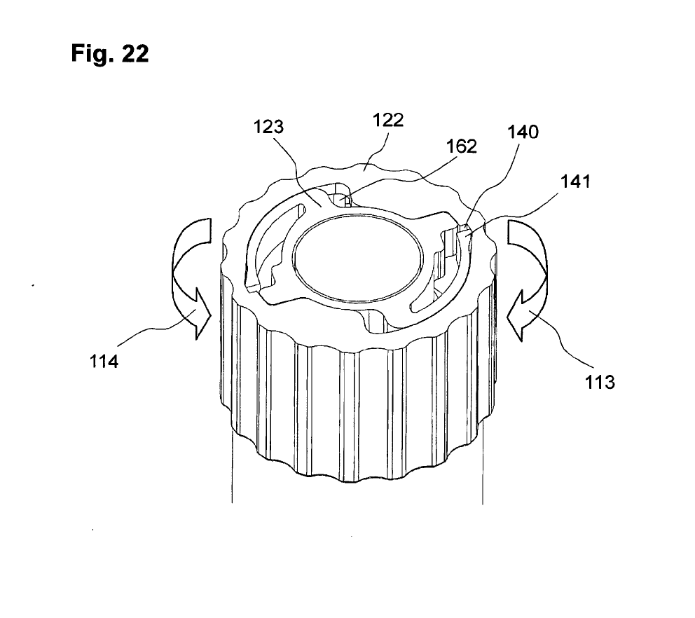

FIG. 22 shows a sectional view of an alternative embodiment of the engagement of the rotation member with the dose setting member,

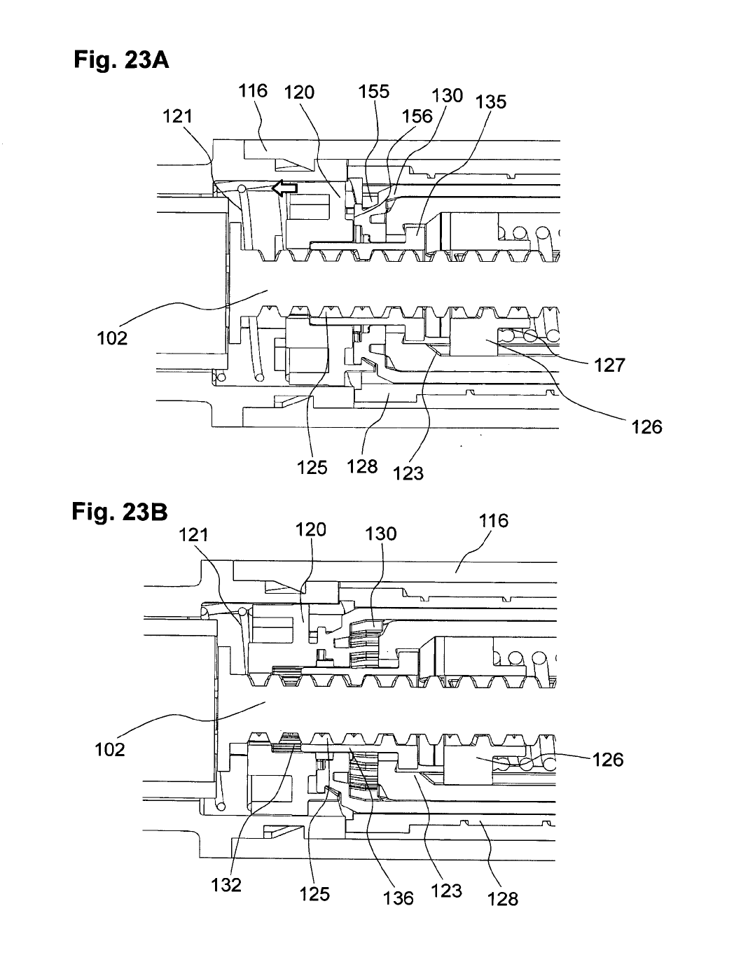

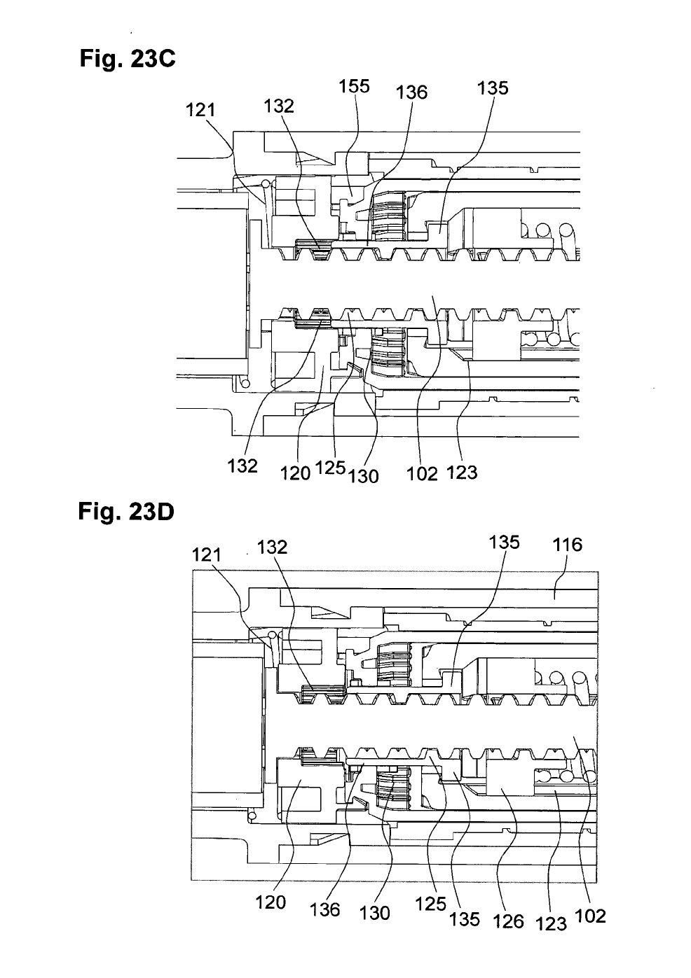

FIGS. 23A to 23D are sectional views, explaining the operation of the mechanism,

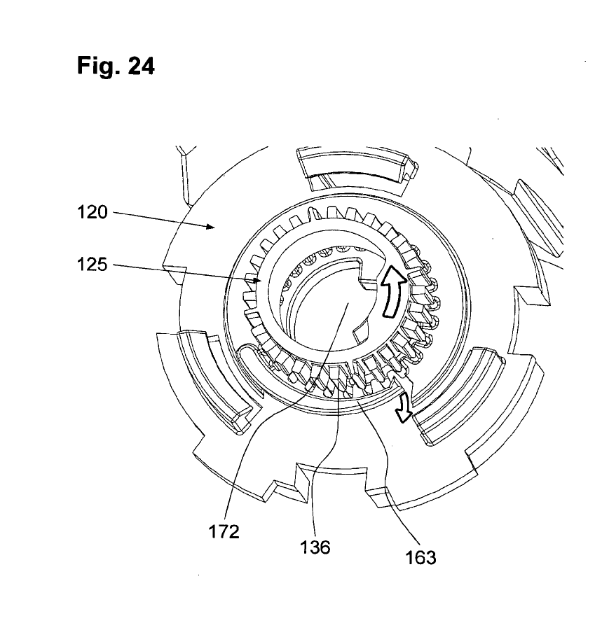

FIG. 24 shows the actuator and the locking member during the dispensing of a dose,

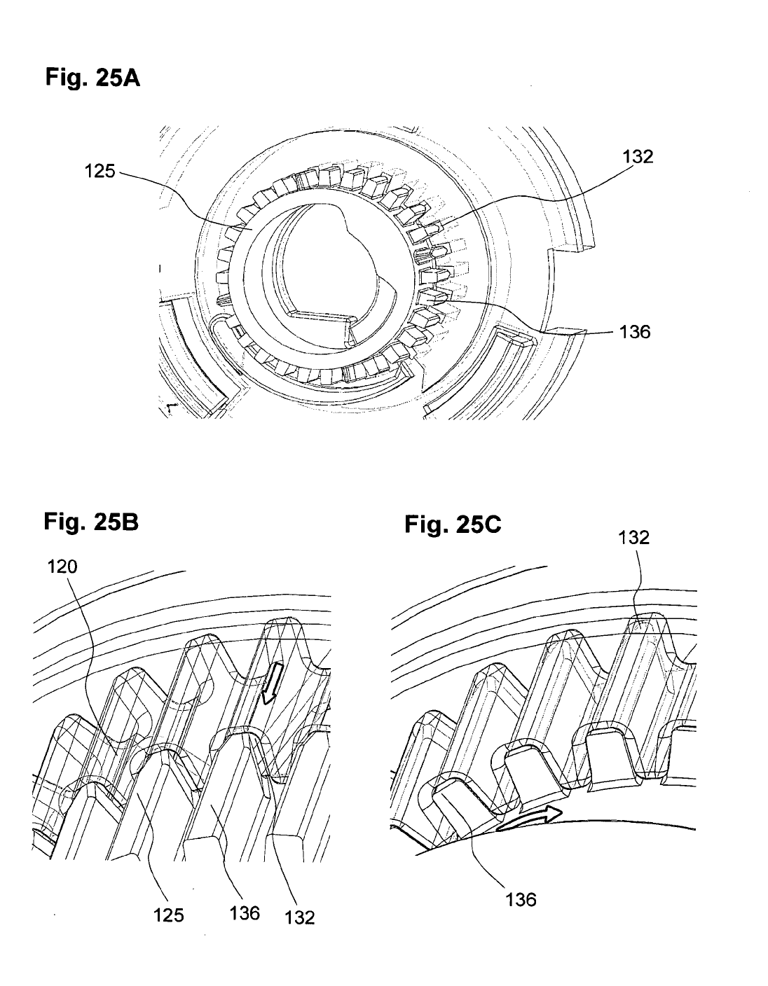

FIGS. 25A to 25C illustrate a re-engagement of the actuator with the locking member after a dose has been dispensed,

FIG. 26 shows an exploded view of a drive assembly for a drug delivery device according to a second embodiment,

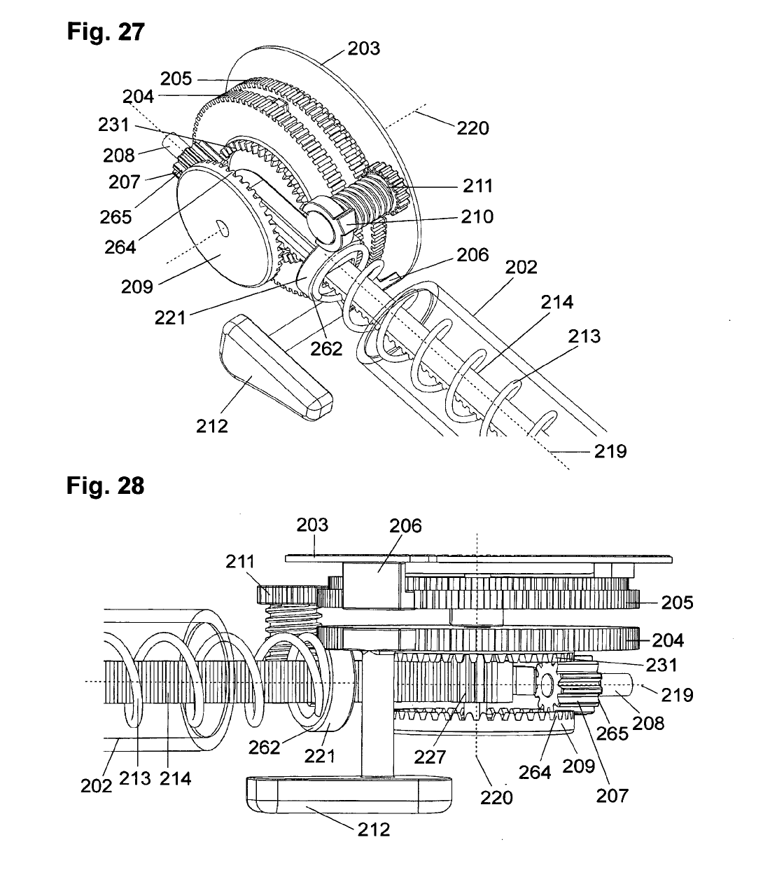

FIG. 27 shows a perspective view of the assembled drive assembly shown in FIG. 26,

FIG. 28 shows another perspective view of the assembled drive assembly shown in FIG. 26,

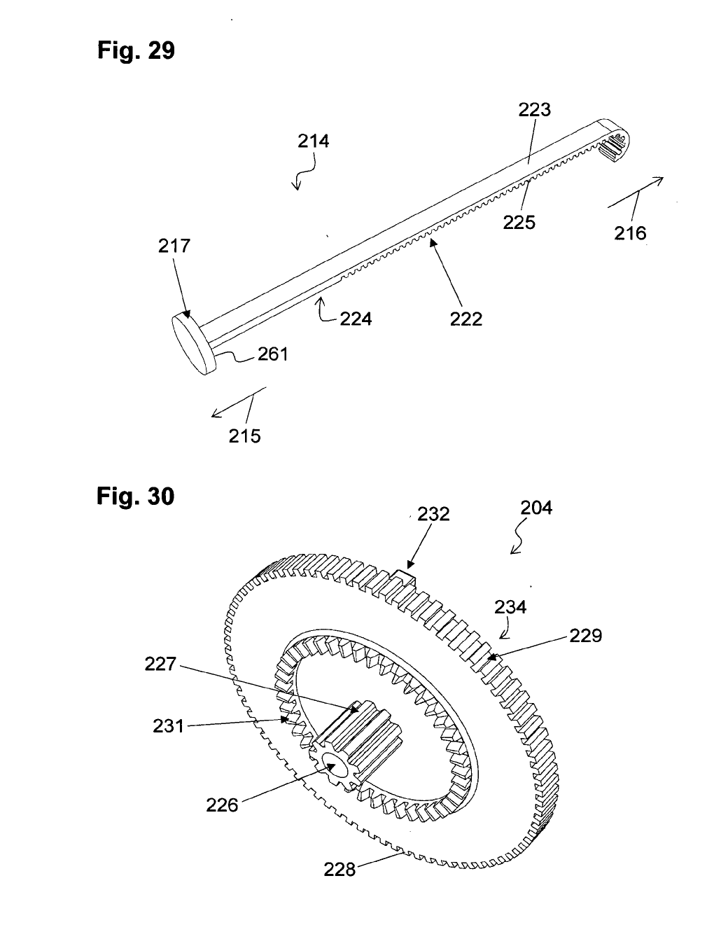

FIG. 29 shows a perspective view of a piston rod,

FIG. 30 shows a perspective view of a drive control member,

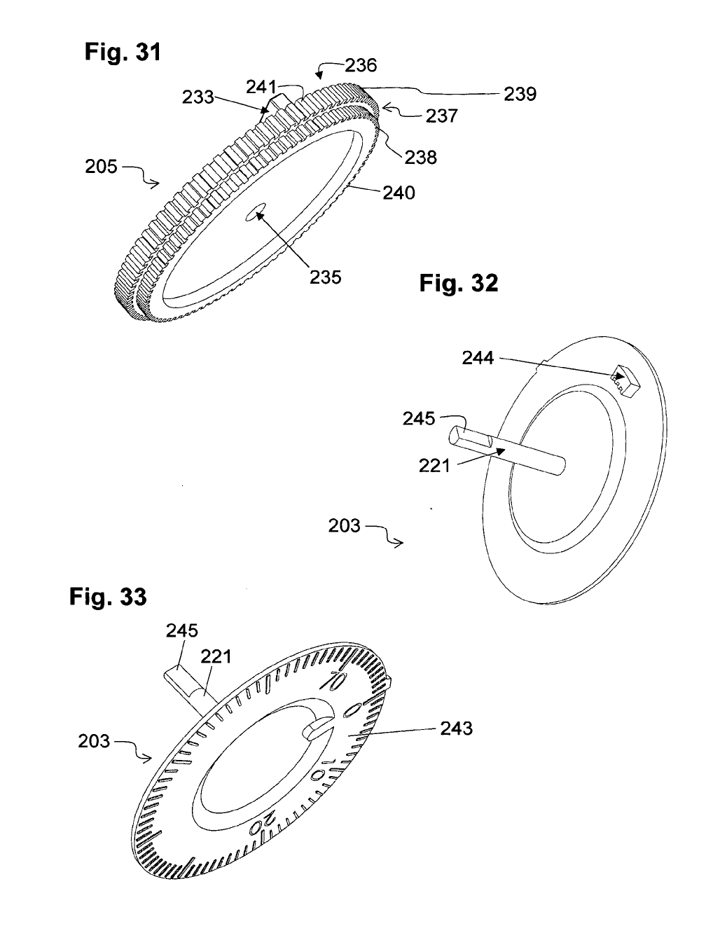

FIG. 31 shows a perspective view of a secondary drive control member,

FIG. 32 shows a perspective view of a dose setting member,

FIG. 33 shows another perspective view of the dose setting member,

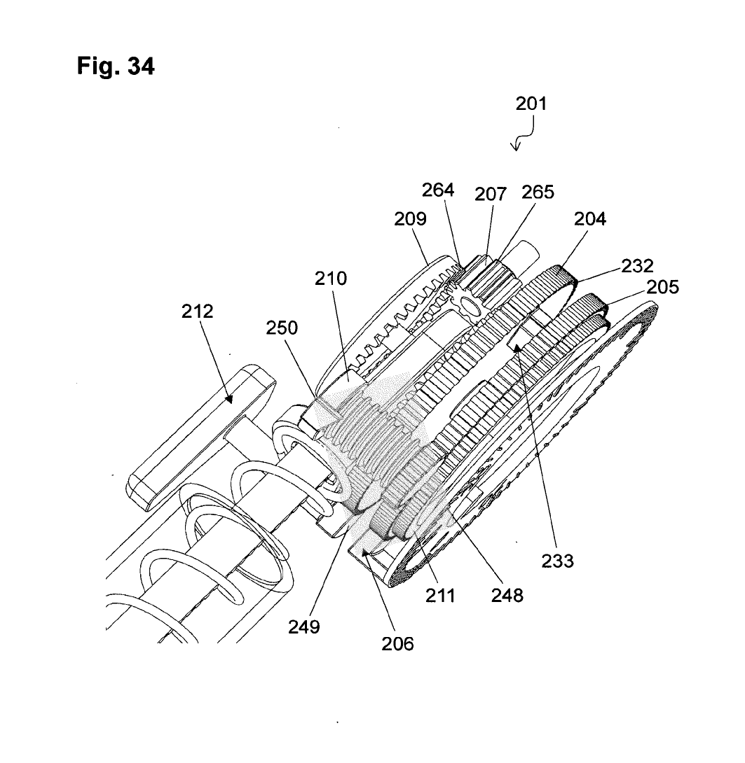

FIG. 34 shows the drive assembly of FIGS. 26 to 28 in a rest state,

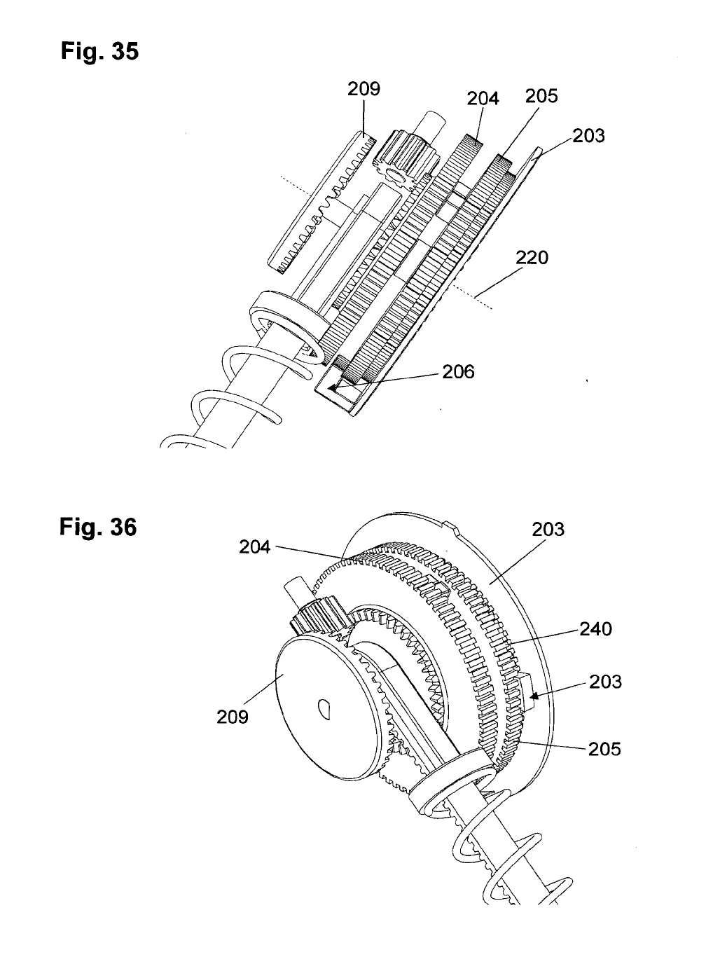

FIG. 35 shows the drive assembly in a ready-to-set state,

FIG. 36 shows the drive assembly in a ready-to-set state from a different perspective,

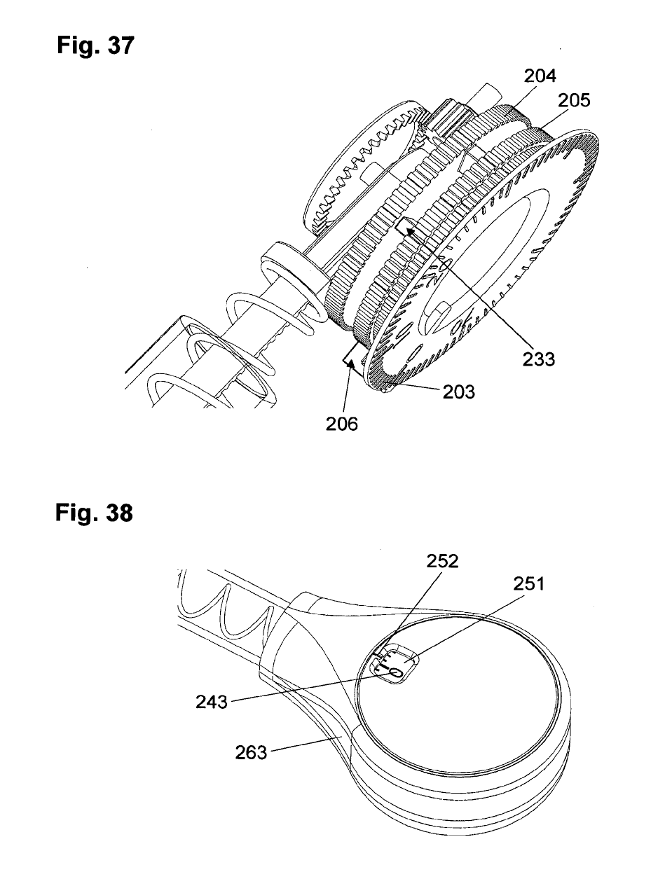

FIG. 37 shows the drive assembly in a dose-set state,

FIG. 38 shows a part of the housing comprising a window,

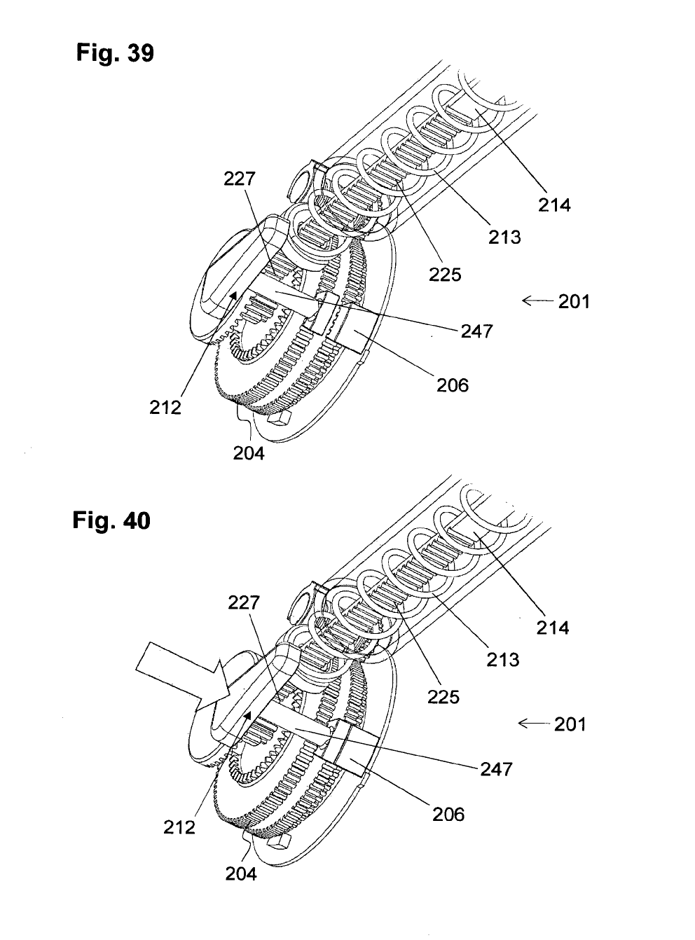

FIG. 39 shows the drive assembly after a dose setting operation is completed,

FIG. 40 shows the drive assembly during initiation of a dose dispensing operation,

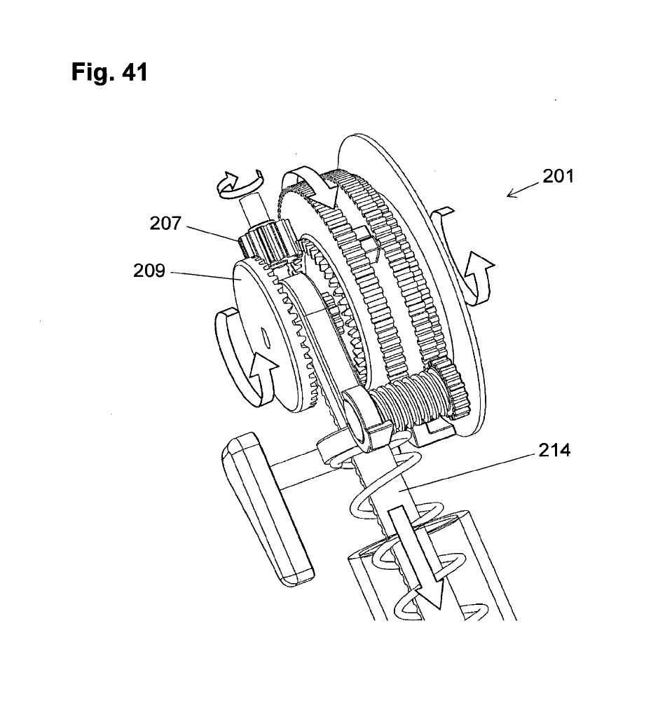

FIG. 41 shows the drive assembly during a dose dispensing operation,

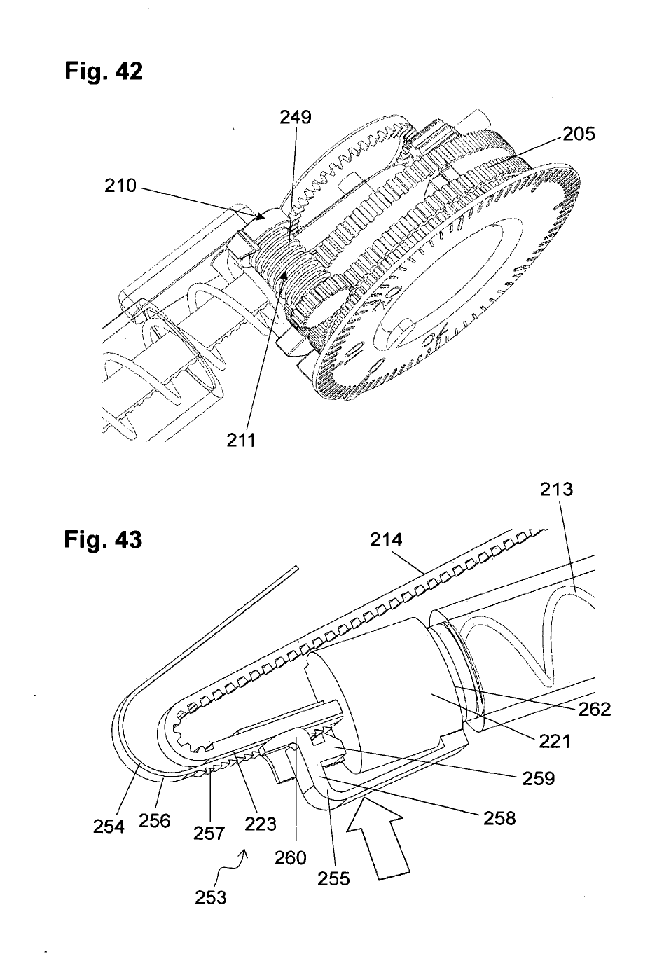

FIG. 42 shows a last dose lockout assembly of the drive assembly,

FIG. 43 shows the drive assembly comprising a safety member wherein the drive assembly is undamaged,

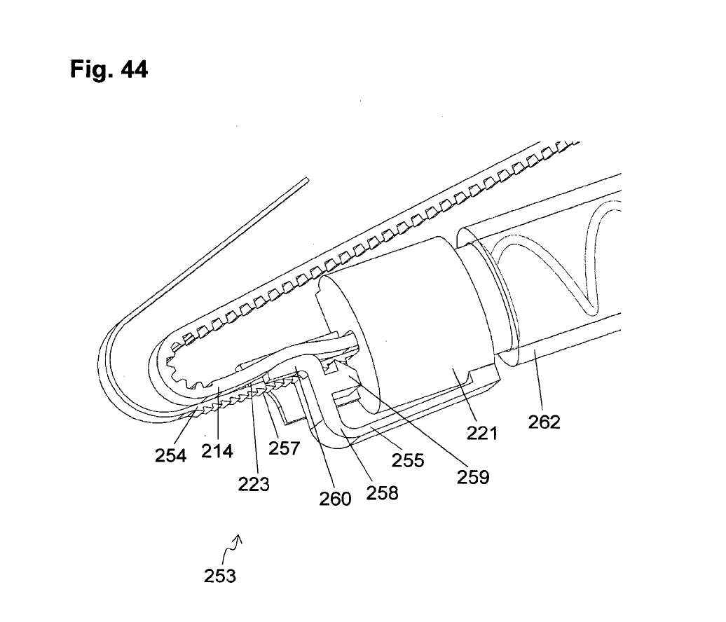

FIG. 44 shows the drive assembly comprising the safety member wherein the drive assembly is damaged,

FIG. 45 shows an exploded view of a drive assembly for a drug delivery device according to a third embodiment,

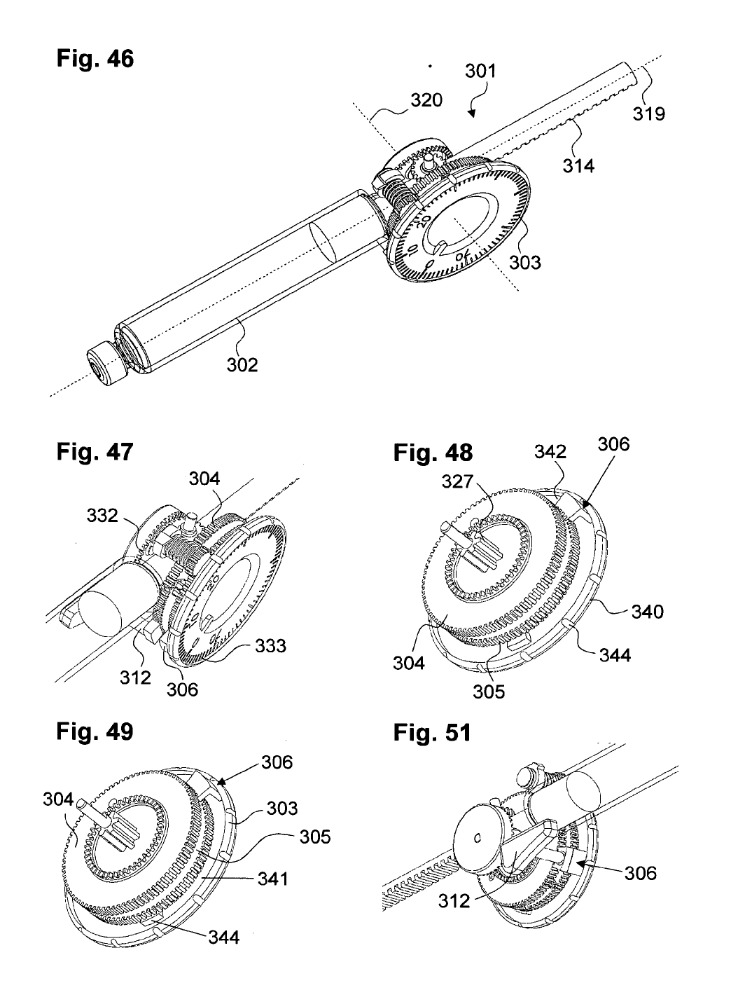

FIG. 46 shows a perspective view of the assembled drive assembly shown in FIG. 45,

FIG. 47 shows the drive assembly in a rest state,

FIG. 48 shows a part of the drive assembly in a rest state,

FIG. 49 shows a part of the drive assembly in a ready-to-set state,

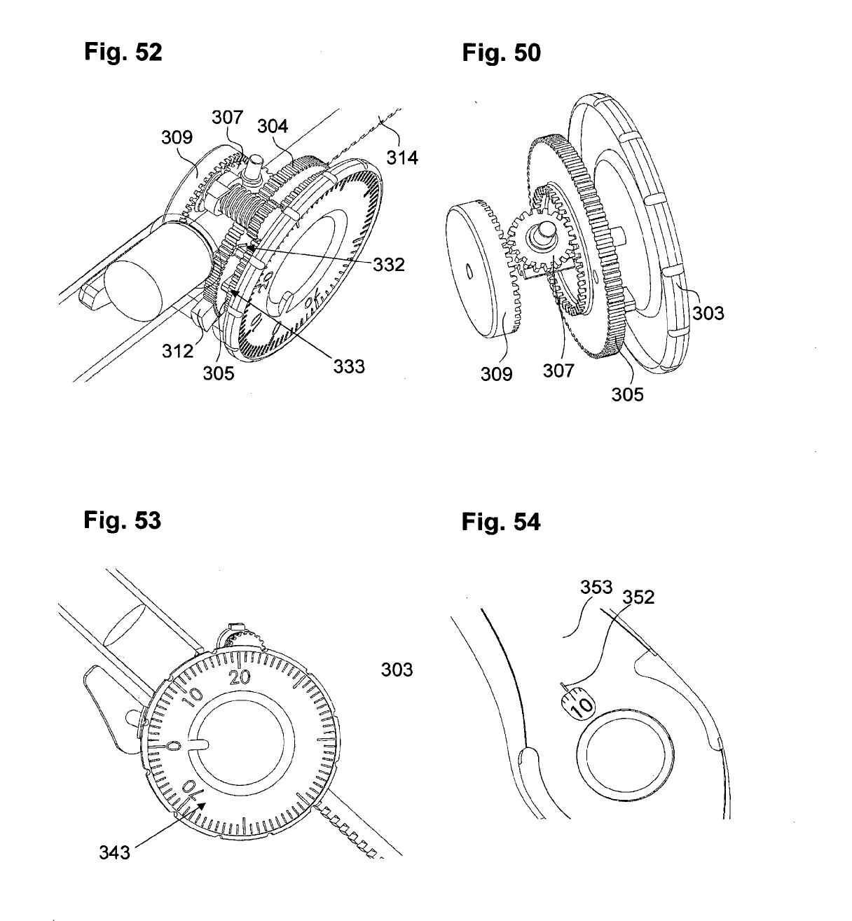

FIG. 50 shows a further part of the drive assembly in a ready-to-set state,

FIG. 51 shows the drive assembly in a ready-to-set state,

FIG. 52 shows a part of the drive assembly in a dose-set state,

FIG. 53 shows a view of an indicator in a dose-set state,

FIG. 54 shows a part of a housing comprising a window in a dose-set state,

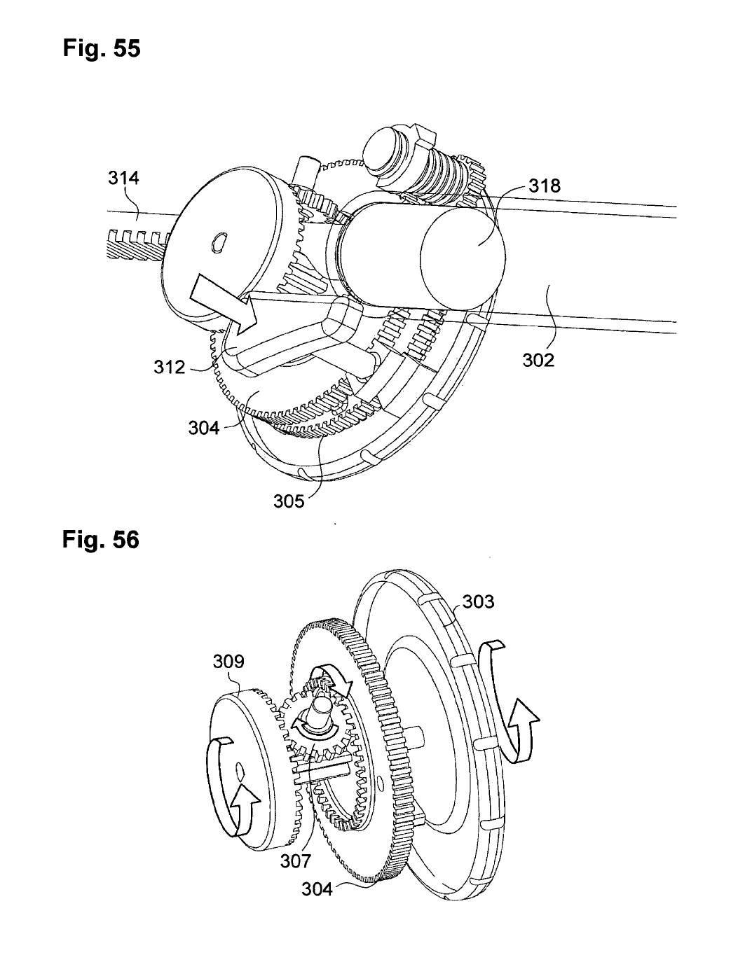

FIG. 55 shows the drive assembly during an initiation of a dose dispensing operation,

FIG. 56 shows a part of the drive assembly during a dose dispensing operation,

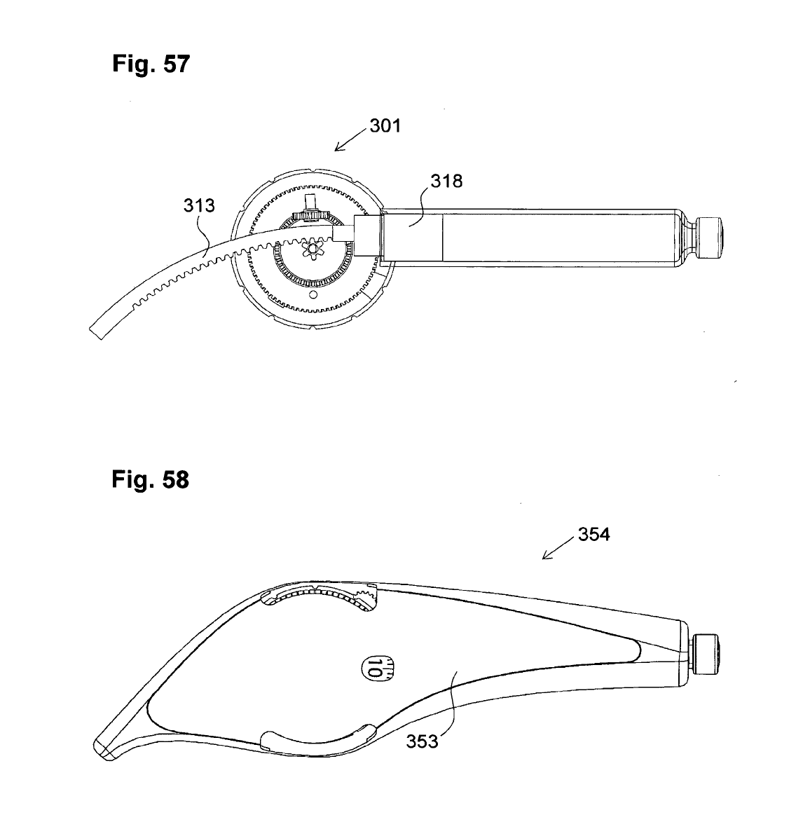

FIG. 57 shows an alternative embodiment of a piston rod for a drive assembly according to the third embodiment,

FIG. 58 shows a drug delivery device according to the alternative embodiment of FIG. 57.

Like elements, elements of the same kind and identically acting elements may be provided with the same reference numerals in the figures.

FIG. 1 shows an exploded view of a drug delivery device 101 comprising a drive assembly according to a first embodiment and an assembly path for the components of the drug delivery device 101. In particular, the drug delivery device 101 is an injection device. The drug delivery device 101 is a variable dose device such that a user can select the size of a dose. The drug delivery device 101 is configured for multiple dose applications. The device can be delivered to a user in a fully assembled condition ready for use. The device has a low part count and is particularly attractive for cost sensitive device applications.

A cartridge 118 is housed within a cartridge holder 117. The cartridge holder 117 is rigidly constrained to a housing 116. An actuator 120 is rotationally constrained to the cartridge holder 117. Between the actuator 120 and the cartridge holder 117, a reset member 121 is arranged. The reset member 121 may be, for example, a spring. An axial force of the reset member 121 is transmitted to and counteracted by the cartridge holder 117. A piston rod 102 is configured to abut a piston 119 which is arranged in the cartridge 118. The piston rod 102 is configured to move the piston 119 in a direction towards a distal end 111 of the device, in order to deliver a medication from the cartridge 118. The piston rod 102 will be described later in more detail.

The drug delivery device 101 further comprises an indicator 128, which is configured to indicate the amount of a set dose of a medication. The indicator 128 may be a number sleeve. The indicator 128 is coupled to a rotation member 123 by means of a coupling member 130. The rotation member 123 may be a sleeve. A window member 147 is placed over the indicator 128. The window member 147 comprises a transparent material. A last dose stop member 124 is engaged with the rotation member 123 by means of a thread. The last dose stop member 124 may be for example a lock nut. The last dose stop member 124 is configured to prevent the setting of a dose which is larger than the remaining amount of medication in the cartridge 118. A locking member 125 and a piston rod nut 126, which will be later described in more detail, are configured to engage with the piston rod 102. The piston rod nut 126 is configured as a drive control member. In particular, the piston rod nut 126 acts on the piston rod 102 for delivering a dose of medication. A spring member 127 is arranged between the piston rod nut 126 and a cap 131.The spring member 127 may be, for example, a coil spring. At a proximal end 112 of the device 101, a dose setting member 122 is arranged.

FIG. 2 shows a sectional view of the drug delivery device 101 in an assembled state. In particular, FIG. 2 shows a drive assembly 180. The dose setting member 122 can be rotated in a dose setting direction 113 in order to set a dose of medication. The dose setting direction 113 may be, for example, a clockwise direction. The dose setting member 122 can be rotated in a dose cancelling direction 114, in order to cancel a set dose of medication. The dose cancelling direction 114 may be, for example, a counter clockwise direction. The drug delivery device 101 permits a cancelling of a dose without any dose of medication being dispensed. When the dose setting member 122 is rotated in a dose setting or dose cancelling direction 113, 114, the rotation member 123 is also rotated due to an engagement of the dose setting member 122 and the rotation member 123, which will be later described in more detail. In particular, when the dose setting member 122 is rotated, the rotation member 123 is rotated with respect to the housing 116. The rotation member 123 is axially fixed with respect to the housing 116. When the rotation member 123 is rotated during the setting of a dose, the piston rod nut 126 is also rotated. The piston rod nut 126 is in threaded engagement with the piston rod 102 and thereby, as the piston rod nut 126 rotates about the piston rod 102, it moves towards a proximal end of the device 112. When the piston rod nut 126 moves towards a proximal end of the device 112, the spring member 127 is compressed between the cap 131 and the piston rod nut 126. In particular, the spring member 127 is compressed to store energy which is charged as a user selects the required dose. This energy is stored until the device is actuated in order to dispense a dose. At this point, the energy stored in the spring member 127 is used to deliver the medication from the cartridge 118 to a user.

The coupling member 130 is arranged concentrically around the distal end of the rotation member 123. During dose setting, the coupling member 130 is engaged with the rotation member 123. Furthermore, the coupling member 130 is engaged with an indicator 128. The indicator 128 is arranged concentrically around the coupling member 130. In particular, the coupling member 130 is rotationally fixed with respect to the rotation member 123 and with respect to the indicator 128 during the setting of a dose. During the dispense of a dose, the coupling member 130 is engaged with and rotationally locked to the locking member 125 and the indicator 128. The coupling member 130 is configured to cause a rotation of the indicator 128 during the setting and dispense of a dose. The coupling member 130 and the indicator 128 will be later described in more detail.

FIG. 3 shows a more detailed view of the rotation member 123, the piston rod 102, the piston rod nut 126 and the locking member 125.

The piston rod nut 126 is in a threaded engagement with the piston rod 102. Furthermore, the piston rod nut 126 is rotationally fixed with respect to the rotation member 123. This is achieved by means of splines 137 of the piston rod nut 126, which engage in axial grooves 154 of the rotation member 123. In an alternative embodiment, the rotation member 123 and the piston rod nut 126 may be coupled by means of splines in the rotation member 123 and grooves in the piston rod nut 126. The piston rod nut 126 is axially moveable with respect to the rotation member 123 along the axial grooves 154 of the rotation member 123. The piston rod 102 is axially and rotationally fixed with respect to the housing 116 of the drug delivery device 101 during the setting and cancelling of a dose. This will be described in more detail in conjunction with FIGS. 7 and 8.

During the setting or cancelling of a dose, the piston rod nut 126 rotates together with the rotation member 123 with respect to the housing 116 of the drug delivery device 101, since the piston rod nut 126 is rotationally fixed with respect to the rotation member 123. Thereby, the piston rod nut 126 rotates with respect to the piston rod 102. Due to the threaded engagement of the piston rod nut 126 and the piston rod 102, the piston rod nut 126 is screwed along the piston rod 102. This results in the piston rod nut 126 moving axially relative to the rotation member 123 and the piston rod 102. In particular, the piston rod nut 126 is moved in a direction towards a proximal end 112 of the device during the setting of a dose, and in a direction towards a distal end of the device during the cancelling of a dose. Furthermore, a locking member 125 is engaged with the piston rod 102. In particular, the locking member 125 is in threaded engagement with the piston rod 102. The thread of the locking member 125 has an opposing helix direction to the piston rod nut 126. During the setting of a dose, the locking member 125 is rotationally fixed with respect to the housing. Thereby, a movement of the piston rod 102 is inhibited by the combination of constraints from the locking member 125 and the spline feature in the actuator 120 during the setting of a dose. During a dispense of a dose, the locking member 125 is enabled to rotate with respect to the housing. In particular, the piston rod 102 overhauls the locking member 125. The torque which is needed to cause the locking member 125 to overhaul the piston rod 102 is provided by the spring member 127.

FIG. 4 shows a more detailed view of the locking member 125. The locking member 125 is configured as a locking nut. The locking member 125 is in a threaded engagement with the piston rod by means of a thread 152. Furthermore, the locking member 125 comprises a plurality of splines 136. The splines 136 are arranged circumferentially around an outer circumference of the locking member 125. The splines 136 are configured to engage with the actuator 120 during the setting of a dose and with the coupling member 130 during the dispense of a dose. The locking member 125 may further comprise extended splines 153. The extended splines 153 may be arranged equally distributed between the splines 136. The ends of the extended splines 153 extend beyond the ends of the splines 136. The ends of the extended splines 153 may be chamfered. By means of the extended splines 153, misalignment tolerances may be diminished. The locking member 125 further comprises a flange 135. By means of the flange 135, the locking member 125 is axially fixed with respect to the housing 116 of the drug delivery device. In particular, the flange 135 abuts an internal surface of a distal end of the rotation member 123. During the setting of a dose, the locking member 125 is rotationally fixed with respect to the housing 116 due to an engagement with the actuator 120, which will be later described in more detail, for example with reference to FIG. 7.

FIG. 5 shows a schematic view of the piston rod 102. The piston rod 102 is a lead-screw. The piston rod 102 comprises a first thread 103 and a second thread 104. The first and the second thread 103, 104 extend over the whole length of the piston rod 102. The first thread 103 and the second thread 104 are counter-handed. The pitch 105 of the first thread 103 is equal to the pitch 106 of the second thread 104. This is to ensure that the indicator 128 is rotated back to its initial position during the dispense of a dose. Since the first thread 103 and the second thread 104 are counter-handed, they intersect each other. In this embodiment the first thread 103 and the second thread 104 are twin start threads. The first thread 103 comprises two thread starts 181, 182. The second thread 104 comprises two thread starts 183, 184. The piston rod 102 comprises at least one axial spline 115. For example, the piston rod 102 may comprise two axial splines 115. The splines 115 run along the entire length of the piston rod 102. In FIG. 5, only one spline 115 is visible. The second spline is arranged opposite to the first spline 115. In this embodiment, the splines 115 are arranged rotationally symmetric. The splines 115 are configured to engage with engagement features 133 of the actuator 120. The locking member 125 is engaged with the first thread 103 of the piston rod 102. The pitch 105 of the first thread 103 engaging the locking member 125 is critical to ensure that an axial force applied to the piston rod 102 generates sufficient torque in the locking member 125 to overcome the thrust bearing friction at an interface between the locking member 125 and the rotation member 123. The piston rod nut 126 is engaged with the second thread 104 of the piston rod 102. The first thread 103 is a right handed thread. The second thread 104 is a left handed thread.

FIG. 6 shows a preferred embodiment of the piston rod 102. The piston rod 102 shown in FIG. 6 is similar to the piston rod 102 shown in FIG. 5, except that a first inner diameter 107 of the first thread 103 is smaller than a second inner diameter 108 of the second thread 104. The first inner diameter 107 is the minor diameter of the first thread 103. The second inner diameter 108 is the minor diameter of the second thread 104. In particular, the first inner diameter 107 may be two times the distance from a main axis 191 of the piston rod 102 to a surface of the pitch 105 of the first thread 103. In particular, the second inner diameter 108 may be two times the distance from a main axis 191 of the piston rod 102 to a surface of the pitch 105 of the second thread 104. In particular, the first thread 103 is cut deeper than the second thread 104. One advantage of a piston rod 102 having a first inner diameter 107 of a first thread 103 which is smaller than a second diameter 108 of a second thread 104 is that there only is a small contact diameter between the first thread 103 and a member being engaged with the first thread 103, in particular the locking member 125. When the locking member 125 overhauls the piston rod 102 during a dispense of a dose, a friction force between the locking member 125 and the piston rod 102 has to be overcome. The smaller the contact diameter between the piston rod 102 and an overhauling element, the smaller the torque generated by this friction force. Therefore, the overhauling torque which has to be provided by the spring member 127 may be kept small compared to an assembly with a piston rod with a larger inner diameter.

Due to the second inner diameter 108 being larger than the first inner diameter 107, the piston rod 102 still has a sufficient mechanical stability.

The piston rod 102 further comprises axial splines, which are not shown in this figure for clarity reasons. The splines are configured as shown in FIG. 5.

FIG. 7 shows the engagement of the actuator 120 with the piston rod 102, the locking member 125 and the coupling member 130 during the setting of a dose or in a non-operating state, when the device is not in use. The actuator 120 is rotationally fixed with respect to the housing 116. The actuator 120 comprises an opening 172, through which the piston rod 102 extends. The actuator 120 is engaged with the locking member 125 by means of a first engagement feature 132 of the actuator 120. The first engagement feature 132 of the actuator 120 may comprise for example splines or teeth, which are arranged at a recess 173 of the actuator 120. The first engagement feature 132 of the actuator 120 engages with the splines 136 of the locking member 125. When the first engagement feature 132 of the actuator 120 is engaged with the splines 136 of the locking member 125, a rotational movement of the locking member 125 with respect to the actuator 120 is inhibited. Since the actuator 120 is rotationally fixed with respect to the housing 116 of the drug delivery device, the locking member 125 is also rotationally fixed with respect to the housing 116, when the first engagement feature 132 of the actuator 120 is engaged with the splines 136 of the locking member 125. Furthermore, the actuator 120 comprises second engagement features 133, which are engaged with the axial splines 115 of the piston rod 102. The second engagement features 133 are arranged at the opening 172 of the actuator 120. The second engagement features 133 of the actuator 120 may be configured as splines or protrusions. Thereby, the piston rod 102 is permanently rotationally fixed with respect to the housing 116 of the drug delivery device. Furthermore, the actuator 120 is engaged with the coupling member 130. In particular, the actuator 120 comprises a snap feature 155 which engages with an engagement feature 156 of the coupling member 130. The snap feature 155 of the actuator 120 may engage the engagement feature 156 of the coupling member 130 during an assembly of the device. Due to this engagement, the coupling member 130 is permanently axially fixed with respect to the actuator 120.

FIG. 8 shows the actuator 120 of FIG. 7 in a cross-sectional view. In this embodiment, the first engagement features 132 of the actuator 120, which are configured to engage with the axial splines 115 of the piston rod 102, are shown. Furthermore, the actuator 120 comprises protrusions 167 which are engaged with grooves in the cartridge holder 117. Thereby, the actuator 120 is rotationally fixed with respect to the cartridge holder 117 and thereby rotationally fixed with respect to the housing 116, since the cartridge holder 117 is rigidly constrained to the housing 116. However, a limited axial travel of the actuator 120 is allowed.

FIGS. 9A and 9B show a drive assembly 180 of the drug delivery device 101 in an assembled state. FIG. 9B shows the drive assembly 180 of the device 101 in a sectional view. In order to set a dose, the dose setting member 122 is rotated in a dose setting direction 113. When the dose setting member 122 is rotated, the rotation member 123 is also rotated. This is because the rotation member 123 is coupled with the dose setting member 122. The coupling of the rotation member 123 and the dose setting member 122 will be later described more detailed with reference to FIGS. 20A and 20B and FIGS. 21A to 21C. Since the piston rod nut 126 is rotationally fixed with respect to the rotation member 123, the piston rod nut 126 rotates with the rotation member 123. Thereby, the piston rod nut 126 is rotated about the piston rod 102 and moves axially along the piston rod 102 towards a proximal end of the device 112. When the piston rod nut 126 is moved towards the proximal end of the device, it compresses the spring member 127. Even when no dose is set, the spring member 127 is lightly compressed, since a minimum force greater than 0 N is required at the piston 119 for all dose sizes. When the dose setting member 122 is rotated in a dose cancelling direction, the piston rod nut 126 is moved towards the distal end of the device and the compression of the spring member 127 is released. The spring member 127 is arranged between the cap 131 and a proximal face 134 of the piston rod nut 126. Arrow 164 indicates the movement of the piston rod nut 126 during the setting of a dose. Arrow 165 indicates the movement of the last dose stop member 124 during the setting of a dose. The last dose stop member 124 and its functionality will be described more detailed with reference to FIGS. 18A to 18C.

When the rotation member 123 is rotated during a setting or a cancelling of a dose, the coupling member 130 and the indicator 128 are also rotated. This is due to an engagement of the coupling member 130 with the rotation member 123, and an engagement of the indicator 128 with the coupling member, which is shown in more detail in FIG. 13. In particular, a rotation of the coupling member 130 results in a rotation and axial translation of the indicator 128. When the indicator 128 is rotated, the numbers shown in an indication window 129 indicate the dose which has been set. A single number on either side of the indication window 129 is also visible to aid in determining the required rotation of the dose setting member 122, as shown in FIG. 10. FIG. 10 shows the proximal part of a drug delivery device 101 with an amount of a set dose being displayed in the indication window 129.

In particular, the coupling member 130 rotates the indicator 128 during both setting and dispensing of a dose to ensure that the correct dose is displayed through the indication window 129. The indication window 129 is a cut-out in the housing of the drug delivery device. Between the indicator 128 and the indication window 129 a window member 147 is arranged. The window member 147 may prevent an intrusion of dust or dirt into the housing of the drug delivery device and may magnify the dose numbers. In this embodiment a dose can be selected between zero and a pre-defined maximum in one unit increments. Any dose can be selected within this range. One unit is for example 0.01 ml.

FIGS. 11A and 11B show the drug delivery device in a state where a dose has been set. In particular, a maximum dose has been set. The maximum is for example 80 units. FIG. 11B shows the device 101 in a sectional view. The amount of the set dose is indicated in the indication window 129. The amount of the set dose is indicated by the indicator 128. The indicator 128 is in its most proximal position. The spring member 127 is compressed by the piston rod nut 126. The last dose stop member 124 has translated axially in a proximal direction, compared to the position of the last dose stop member 124 shown in FIG. 9B. When the actuator 120 is actuated by a user, as indicated by an arrow in FIG. 11A, in particular moved in the distal direction, the set dose of medication is delivered from the drug delivery device. When the actuator 120 is actuated, the locking member 125 is disengaged from the actuator 120. This mechanism will be later described in more detail with reference to FIGS. 22A to 22D. When the locking member 125 is disengaged from the actuator 120, the locking member 125 is enabled to rotate with respect to the housing 116. When the locking member 125 is enabled to rotate, the piston rod 102 is enabled to axially move with respect to the housing 116. During dispense, the locking member 125 is driven rotationally in the opposing direction to the direction of the rotation member 123 when setting a dose and, therefore, turns the indicator 128 backwards to reduce the value of the dose displayed.

The piston rod 102 moves in a direction towards a distal end 111 of the drug delivery device when a dose has been set and the actuator 120 is actuated. In particular, the spring member 127 exerts a force on the proximal face 134 of the piston rod nut 126. This force moves the piston rod 102 towards a distal end of the device. In particular, the piston rod 102 moves axially, but does not rotate with respect to the housing 116. When the piston rod 102 is moved in a direction towards a distal end of the device, the locking member 125 overhauls against a thread of the piston rod 102. During the dispensing of a medication, the indicator 128 is moved back to its initial position.

FIGS. 12A and 12B show the drug delivery device 101 in a condition when a dose of medication has been delivered from the device. All components besides the piston rod 102, the last dose stop member 124, the dose setting member 122 and the rotation member 123 are in their initial position. In particular, the indicator 128 is in its initial position, such that the number “0” is shown in the indication window 129.

FIG. 13 shows the coupling member 130 engaged with the indicator 128 in a detailed view. In particular, the indicator 128 is rotationally constrained to the coupling member 130. This is achieved by engagement means 143 of the coupling member 130 being engaged with engagement means 144 of the indicator 128. For example, the engagement means 144 of the indicator 128 may be splines which engage with corresponding grooves in the coupling member 130. The indicator 128 and the coupling member 130 remain in engagement throughout the range of axial travel of the indicator 128.

FIG. 14 shows a schematic view of the indicator 128 and the window member 147, which is arranged concentrically around the indicator 128. The indicator 128 is printed with a helical path of numbers, the pitch of the helix matching the pitch of a thread connecting the indicator 128 and the window member 147. The thread 148 connecting the indicator 128 and the window member 147 is shown in FIG. 16. The number of the indicator 128 that is visible through the window member 147 corresponds to the set dose. The window member 147 comprises a magnifying element to make the numbers on the indicator 128 more distinct for a user. The indicator 128 comprises at least one maximum dose abutment 145. The window member 147 comprises at least one maximum dose abutment 146. The maximum dose abutment 146 inhibits the setting of a dose beyond a specified amount. The indicator 128 further comprises at least one stop feature 170. The stop feature 170 is configured to abut a stop feature 171 of the cartridge holder 117 when a set dose has been completely dispensed. In particular, the stop feature 170 acts as an end of dispense stop and dial stop.

A section of the cartridge holder 117 and the indicator 128 are shown in FIGS. 15A and 15B. In FIG. 15A, the stop feature 170 of the indicator 128 approaches the stop feature 171 of the cartridge holder 117 during a dispense of a dose. In FIG. 15B, the stop feature 170 of the indicator 128 abuts the stop feature 171 of the cartridge holder 117. When the stop feature 170 of the indicator 128 abuts the stop feature 171 of the cartridge holder 117 further rotation of the indicator 128 is inhibited. Accordingly, the cartridge holder 117 provides a rotational stop for the indicator 128 at the end of dose condition. Furthermore, when a rotation of the indicator 128 is inhibited, a rotation of the coupling member 130 is also inhibited. When a rotation of the coupling member 130 is inhibited, a rotation of the locking member 125 is inhibited. Thereby, the dispense of a dose of medication is inhibited.

In FIG. 16, a portion of the window member 147 is shown in more detail. The window member 147 is configured to be connected to the indicator 128 via a thread 148. Furthermore, the window member 147 comprises engagement means 158. The engagement means 158 of the window member 147 are configured to engage with the housing 116. Thereby, a rotation of the window member 147 relative to the housing 116 is inhibited. In particular, the window member 147 is rigidly constrained to the housing 116. For example, the engagement means 158 of the window member 128 may be splines. Alternatively, the engagement means 158 may be grooves.

FIGS. 17A to 17C show the window member 147 and the indicator 128 in three different states during the setting of a dose. The indicator 128 is threaded to the window member 147 such that rotation of the indicator 128 by the coupling member 130 results in a rotation and axial translation of the indicator 128 with respect to the window member 147. During the setting of a dose, the maximum dose abutment 145 of the indicator 128 approaches the maximum dose abutment 146 of the window member 147, as shown in FIGS. 17A and 17B. When a maximum dose has been set, the maximum dose abutment 145 of the indicator 128 abuts the maximum dose abutment 146 of the window member 147, as shown in FIG. 17C. Thereby, a further rotation of the indicator 128 is inhibited. Thereby, the setting of a dose beyond a maximum dose is inhibited. The maximum dose is visible through the window member 147.

FIGS. 18A to 18C show the position of the last dose stop member 124 relative to the rotation member 123 in three different states of the device. The rotation member 123 acts as a last dose stop drive member 190. The number of permissible rotations of the rotation member 123 relative to the last dose stop member 124 is determined by the capacity of the cartridge 118. In particular, a movement of the rotation member 123 results in a movement of the last dose stop member 124. The last dose stop member 124 is rotationally fixed but axially movable with respect to the housing 116. This is achieved by means of at least one protrusion 176 of the last dose stop member 124, which is configured to engage with the housing 116, for example with at least one axial groove 177 (see FIG. 2) of the housing 116. The last dose stop member 124 is engaged with the rotation member 123 by means of a thread 161. The last dose stop member 124 comprises a last dose stop member abutment 159. FIG. 18A shows the last dose stop member 124 in a position before any dose has been set. When the rotation member 123 is rotated in a dose setting direction 113 the last dose stop member 124 moves along the rotation member 123 towards a proximal end of the device. When only a small amount of medication is left in a cartridge, a last dose stop face 160 of the rotation member 123 approaches the last dose stop face 159 of the last dose stop member 124 as can be seen in FIG. 18B. When the last dose stop face 160 of the rotation member abuts the last dose stop face 159 of the last dose stop member 124, as shown in FIG. 18C, a further setting of a dose is inhibited. This is because further rotation of the rotation member 123 in a dose setting direction 113 is inhibited. Thereby, the setting of a dose which is larger than a dose of medication remaining in the cartridge is inhibited. Yet, the cancelling of a set dose of medication is still possible by rotating the rotation member 123 in a dose cancelling direction 114. When the rotation member 123 is rotated in the dose cancelling direction 114, the last dose stop member 124 is moved towards the distal end 111 of the device.

FIG. 19 shows a section through the proximal end of the drug delivery device. This section shows an axial constraint between the housing 116 and the dose setting member 122 by means of a protrusion 169 in the dose setting member 122. Furthermore, the arrangement of the cap 131 is shown. The cap 131 is constrained within the dose setting member 122. The cap 131 contacts a distal surface of the dose setting member 122 via a small diameter bearing 178. Through this interface, a force of the piston 119 acting on the piston rod 102 is transmitted to and counteracted by the housing 116. In particular, the small diameter bearing 178 provides a bearing for the rotation member 123. Furthermore, the spring member 127 contacts the cap 131. The cap 131 is axially fixed to the rotation member 123 via constraint features 175 which engage with the rotation member 123.

FIG. 20 shows the engagement of the rotation member 123 with the dose setting member 122. The rotation member 123 is mounted on the cap 131. The dose setting member 122 comprises at least one, for example two ratchet features 140. The ratchet features 140 are configured as indentations in the dose setting member 122. The rotation member 123 comprises at least one, for example two ratchet arms 141. The ratchet arms 141 of the rotation member 123 engage with the ratchet features 140 of the dose setting member 122. When the dose setting member 122 is not rotated, for example during the dispense of a dose, or when a dose has been set, the ratchet arms 141 abut the ratchet features 140 of the dose setting member 122. This is due to a torque on the rotation member which derives from the spring member 127. In particular, the torque from the spring member 127 is transmitted to and counteracted by the drive member 122. During the cancelling of a dose, there may be a gap between the ratchet arms 141 and the ratchet features 140 for a short duration. In particular, the ratchet arms 141 may disengage from the ratchet features 140 for a short duration during the cancelling of a dose. The ratchet arms 141 of the rotation member 123 are furthermore in engagement with a housing ratchet feature 142 of a housing of the drug delivery device. The housing ratchet feature 142 may be for example a plurality of teeth or indentations located at an inner circumference of the housing 116.

The ratchet interface between the dose setting member 122 and the housing 116 ensures that the torque from the spring member 127 does not return the device to a zero-unit position when a user releases the dose setting member 122 after a dose has been set. The zero-unit position is a position where no unit of a dose is set.

FIGS. 21A to 21C show the dose setting member 122 and the rotation member 123 according to FIG. 20, in particular the ratchet feature 140 of the dose setting member 122 and the ratchet arms 141 of the rotation member 123 in three different states.

FIG. 21A shows the engagement of the dose setting member 122 and the rotation member 123 in a state during the setting of a dose. When the dose setting member 122 is rotated in a dose setting direction 113, the rotation member 123 is rotated with it due to the engagement of the ratchet arms 141 with the ratchet feature 140 of the dose setting member 122. In particular, the dose setting member 122 acts on a radial face 179 of the ratchet arm 141 and rotates the rotation member 123 directly, forcing it to engage with a subsequent indentation or tooth of the housing ratchet feature 142. The ratchet feature 140 is configured as an indentation in the dose setting member 122. An inner circumference of the dose setting member 122 slightly extends over the housing ratchet feature 142 of the housing in a direction towards a longitudinal axis of the drug delivery device. In particular, the ratchet feature 140 of the dose setting member 122 is enlarged with respect to the housing ratchet feature 142. Therefore, the ratchet arm 141 of the rotation member 123 can disengage from the housing ratchet feature 142 when the dose setting member 122 is rotated in a dose setting direction 113, but can not disengage from the ratchet feature 140 of the dose setting member 122. A ramp angle of the ratchet feature 140 is reduced in order to ensure that the ratchet arm 141 fully reengages with the housing ratchet feature 142 before it abuts the ratchet feature 140. This is to prevent a user from experiencing shock load through the dose setting member 122. When the ratchet arm 141 reengages with the housing ratchet feature 142, an audible feedback may be given to a user. Furthermore, the housing ratchet feature 142 inhibits an unintended rotation of the rotation member 123 in a dose cancelling direction.

FIG. 21B shows the rotation member 123 and the dose setting member 122 in a condition when the dose setting member 122 is not rotating. This state may temporarily also occur during a rotation of the dose setting member 122. In this state, the ratchet arms 141 of the rotation member is fully engaged with the ratchet feature 140 of the dose setting member and with the housing ratchet feature 142.

FIG. 21C shows the rotation member 123 and the dose setting member 122 during the cancelling of a dose. When the dose setting member 122 is rotated in a dose cancelling direction 114, the ratchet arm 141 is temporally disengaged from the ratchet feature 140 of the dose setting member 122. Furthermore, the ratchet arm 141 is disengaged from the housing ratchet feature 142. This is because the ratchet arm 141 is deflected in a radial inward direction by the dose setting member 122. This is achieved by the dose setting member 122 acting on a sloped face 185 of the ratchet arm 141. Due to the torque acting on the rotation member 123 by the spring member 127, the rotation member is rotated in a dose cancelling direction until the ratchet arms 141 reengage with the ratchet feature 140 of the dose setting member. The dose setting member 122 can now be turned in either direction to increase or decrease the set dose.

FIG. 22 shows an alternative embodiment of the dose setting member 122. In this embodiment, a ratchet arm spring force required is reduced. Thereby, a dialling torque is reduced. This embodiment comprises additional engagement features 162, which engage with an abutment of the rotation member 123. The additional engagement features 162 are configured as lugs. The dose setting member 122 comprises two lugs. The strength of the engagement between the dose setting member 122 and the rotation member 123 is increased, when the dose setting member 122 is rotated in a dose setting direction 113. Furthermore, this engagement can also drive the rotation member 123 in the dose cancelling direction 114. The engagement features 162 of the dose setting member 122 are configured to rotate the rotation member 123 in a dose setting direction 113. Thereby, the ratchet arms 141 of the rotation member 123 are unburdened during the setting of a dose. Thereby, the ratchet arms 141 may be prevented from being damaged, for example under load applied by the user when a dial stop is engaged, and can be optimally designed to perform the single function of resisting the spring torque. In particular, the ratchet arms 141 do not transfer the rotation of the dose setting member 122 to the rotation member 123. The rotation of the rotation member 123 is only achieved by means of the engagement features 162. An additional benefit is removal of a sliding friction interface between the dose setting member 122 and the ratchet arm 141.

FIGS. 23A to 23D explain the operation of the mechanism when the actuator 120 is actuated and a dose is dispensed. The force required for actuating the actuator 120 and the distance which it has to move are small, providing a significant ergonomic advantage, particularly for such users with impaired dexterity.

FIG. 23A shows the mechanism before the actuator 120 is actuated. The locking member 125 is engaged with the actuator 120 and thereby rotationally fixed with respect to the housing 116 of the drug delivery device. The coupling member 130 is rotationally fixed with respect to the rotation member 123 due to an engagement of the coupling member 130 with the rotation member 123. Since the locking member 125 is in its locking state due to its engagement with the actuator 120, the piston rod 102 is axially and rotationally fixed with respect to the housing. In particular, the locking member 125 is non-rotatable with respect to the piston rod 102. When the actuator 120 is moved towards the distal end of the device, as shown in FIG. 23B, the coupling member 130 is moved with the actuator 120. This is due to the engagement of the snap feature 155 of the actuator with the engagement feature 156 of the coupling member 130. The locking member 125 remains in its axial position due to the flange 135 of the locking member 125 abutting a surface of the rotation member 123.

When the actuator 120 is further moved towards a distal direction as shown in FIG. 23B the coupling member 130 is pulled into engagement with the splines 136 of the locking member 125. Thereby, the coupling member 130 is rotationally fixed with respect to the locking member 125. When the actuator 120 has reached the position shown in FIG. 23C, the coupling member 130 is completely disengaged from the rotation member 123. When the actuator 120 has reached the position shown in FIG. 23D, the engagement between the locking member 125 and the actuator 120 is released. In particular, the first engagement feature 132 of the actuator 120 is disengaged from the splines 136 of the locking member 125.When the locking member 125 is completely disengaged from the actuator 120 it is enabled to rotate with respect to the housing 116. Thereby, the piston rod 102 is enabled to axially move with respect to the housing 116. When the piston rod 102 is enabled to move, in particular when the locking member 125 is enabled to rotate, a force of the spring member 127 is released. In particular, the spring member 127 is enabled to relax. In particular, the piston rod 102 is moved in a distal direction by the force of the spring member 127. In particular, the spring member exerts a force on the piston rod nut 126, thereby moving the piston rod nut 126 and together with it the piston rod 102. Thereby, the locking member 125 overhauls against a thread of the piston rod 102 as the piston rod 102 is moved distally.

When the spring member 127 acts on the proximal surface of the piston rod nut 126, thereby moving the piston rod 102 in a distal direction, the flange 135 of the locking member 125 is pressed against the inner surface of the rotation member 123. Thereby, the rotation of the locking member 125 is impeded by friction. If the torque which is needed to overhaul the piston rod 102 is reduced, the force of the spring member 127 acting on the piston rod nut 126 will also reduce, and the locking member 125 is pressed against the inner surface of the rotation member 123 with less force. Thereby, the frictional losses at the interface between the flange 135 of the locking member 125 and the inner surface of the rotation member 123 can be reduced. This can be achieved by using a piston rod 102 wherein the inner diameter 107 of the first thread 103 is smaller than the inner diameter 108 of the second thread 104. Such a piston rod 102 is shown in FIG. 6.

Since the coupling member 130 is in engagement with the splines 136 of the locking member 125, the coupling member 130 rotates together with the locking member 125. In particular, the coupling member rotates in a direction which is counter to the rotation of the coupling member 130 during the setting of a dose. Thereby, the coupling member 130 rotates the indicator 128 back to its initial position. Accordingly, the locking member 125 acts as a reversing member, as its movement causes a rotation of the indicator 128 via the coupling member 130 back to its initial position.

The reset member 121 reacts on a proximal face of the cartridge holder 117. It provides a return force in the proximal direction to return the actuator 120 to its initial position when a user releases the actuator 120.

FIG. 24 shows the actuator 120 and the locking member 125 during the dispensing of a dose. In this state, the locking member 125 is enabled to rotate with respect to a housing of the drug delivery device, in particular with respect to the actuator 120. The actuator 120 comprises a feedback feature 163, which may be, for example, a flexible arm. The feedback feature 163 may be lightly engaged with the splines 136 of the locking member 125. When the locking member 125 rotates, in particular when the splines 136 pass the feedback feature 163, the feedback feature 163 is deflected in a radially outward direction, in particular in a direction away from a longitudinal axis of the drug delivery device. When one spline passes the feedback feature 163, an audible click is produced. In particular, a click is produced when the feedback feature 163 rapidly returns to its undeflected position. Each click corresponds to the dispense of a single unit. This is because the number of splines 136 on the locking member 125 is equal to the number of units dispensed during one rotation of the locking member 125.

FIGS. 25A to 25C illustrate a reengagement of the actuator 120 with the locking member 125 after a dose has been dispensed and the actuator 120 has been released. When the actuator 120 is released, the first engagement feature 132 of the actuator 120 reengages with the splines 136 of the locking member 125. The engagement features 132 of the actuator 120 are angled such that during a reengagement the locking member 125 is turned against the torque being produced by the spring member 127. Thereby, the locking member 125 is wound back a small distance. Thereby, the piston rod is retracted a small distance. This back-winding of the locking member 125 removes the effect of clearances within the mechanism, which are a result of manufacturing tolerances. These tolerances could otherwise lead to slight advancement of the piston rod and a dispense of some medication during selection of a subsequent dose. The back-winding of the locking member 125 retracts the piston rod 102 and ensures that the locking member 125 is acting as the dispense stop in place of the indicator 128.

FIG. 26 shows an exploded view of a drive assembly 201 for a drug delivery device according to a second embodiment. The drive assembly 201 can be operated to deliver variable doses of a medicinal product from a cartridge 202, via a needle (not shown).

The drive assembly 201 comprises a dose setting member 203, a drive control member 204, a secondary drive control member 205, a drive control member stop 206, a reversing member 207, a reversing member shaft 208, a coupling member 209, a last dose stop member 210, a last dose stop drive member 211, an actuator 212, a spring member 213 and a piston rod 214. The components of the drive assembly 201 will be discussed in detail in the following. The drive assembly 201 is configured to move a piston 218 further into the cartridge 202 in a distal direction 215.

The piston rod 214 comprises a bearing 217 arranged at the distal end of the piston rod 214. The bearing 217 is adapted to provide a force on the piston 218 arranged in the cartridge 202 such that the piston 218 is moved in the distal direction 215 further into the cartridge 202. Thereby, a medicinal product is expelled from the cartridge 202.

The drive assembly 201 comprises a main axis 219. The main axis 219 of the drive assembly 201 corresponds to a longitudinal axis of the cartridge 202. The piston rod 214, the spring member 213, the reversing member 207 and the reversing member shaft 208 are located on the main axis 219 of the drive assembly 201.

Further, the drive assembly 201 defines a second axis 220. The second axis 220 is perpendicular to the main axis 219. In particular, the second axis 220 is defined by a shaft 221 of the dose setting member 203. In the drive assembly 201, the dose setting member 203, the secondary drive control member 205, the drive control member 204 and the coupling member 209 are arranged coaxially on the second axis 220.

The drive assembly 201 is configured to be located in a housing of the drug delivery device. In FIG. 26, the housing is not fully represented for clarity. However, in FIG. 26, a housing part 221 is shown.

Moreover, the drive assembly 201 may comprise a safety member which is not shown in FIG. 26. The safety member may be configured to prevent a movement of the piston rod 214 when the drive assembly 201 is damaged. The safety member will be discussed in detail later on.

FIGS. 27 and 28 show perspective views of the assembled drive assembly 201. In particular, the main axis 219 and the second axis 220 are shown in FIGS. 27 and 28.

FIG. 29 shows the piston rod 214. The piston rod 214 comprises the bearing 217 at its distal end. The bearing 217 is integrally formed with the piston rod 214. In particular, the bearing 217 forms a first spring seat 261. In the assembled drive assembly 201, one end of the spring member 213 abuts the first spring seat 261.

Moreover, the piston rod 214 is flexible such that it can be wound around other elements of the drive assembly 201. In particular, as shown in FIG. 28, the piston rod 214 is partially wound around an inner small diameter pinion gear 227 of the drive control member 204.

The piston rod 214 comprises a main part 222 extending in the proximal direction 216 from the bearing 217. The main part 222 has an upper main surface 223 and a lower main surface 224. In the assembled drive assembly 201, as shown in FIGS. 27 and 28, the lower main surface 224 of the piston rod 214 faces towards the inner small diameter pinion gear 227 of the drive control member 204. Further, in the assembled drive assembly 201, the upper main surface 223 of the piston rod 214 faces away from the inner small diameter pinion gear 227 of the drive control member 204.

The piston rod 214 comprises teeth 225. The teeth 225 extend along the main part 222 of the piston rod 214. In particular, the teeth 225 cover more than half of the lower main surface 224 of the main part 222 of the piston rod 214. The teeth 225 are adapted to engage the piston rod 214 with the inner small diameter pinion gear 227 of the drive control member 204. In particular, the teeth 225 are configured to prevent the piston rod 214 from moving, unless the drive control member 204 is enabled to rotate.

The spring member 213 comprises a coil spring. During assembly of the drive assembly 201, the spring member 213 is compressed between the first spring seat 261 and a second spring seat 262. The housing part 221 forms the second spring seat 262. A second end of the spring member 213 abuts the second spring seat 262, as shown in FIGS. 27 and 28.

Further, the spring member 213 is configured such that it is capable of delivering all the required doses from the cartridge 202 without being further compressed during a dose setting or a dose dispensing operation. In particular, in its compressed state, the spring member 213 exerts a force on the first spring seat 261 of the piston rod 214. Accordingly, when a locking of the piston rod 214 is released, this force tends to move the piston rod 214 in the distal direction 215. In particular, the spring member 213 exerts the force on the first spring seat 261 formed by the bearing 217 which moves the piston 218 in the distal direction 215 and results in expelling a medicinal product from the cartridge 202.

FIG. 30 shows the drive control member 204. The drive control member 204 runs on the shaft 221 which is an integral part of the dose setting member 203. For this purpose, the drive control member 204 comprises a through hole 226 in which the shaft 221 of the dose setting member 203 is arranged. The drive control member 204 comprises the inner small diameter pinion gear 227. The inner small diameter pinion gear 227 is located on an outer face 228 of the drive control member 204 which faces away from the dose setting member 203 in the assembled drive assembly 201. The inner small diameter pinion gear 227 is in toothed engagement with the piston rod 214, in particular with the teeth 225 of the piston rod 214.

Further, the drive control member 204 comprises teeth 229 located on its outer perimeter. The teeth 229 face in a direction away from the second axis 220. The teeth 229 arranged at the outer perimeter of the drive control member 204 are configured to engage with splines 230 on the actuator 212. The splines 230 in the actuator 212 are shown in FIG. 26. When the teeth 229 are engaged with the splines 230 of the actuator 212, the drive control member 204 is prevented from rotating relative to the actuator 212 and thereby also from rotating relative to the housing of the drug delivery device. However, if a user depresses the actuator 212, the drive control member 204 disengages from the actuator 212 and is enabled to rotate.

The drive control member 204 further comprises a set of crown gear teeth 231 which are arranged at its outer face 228 facing away from the dose setting member 203 in the assembled drive assembly 201. The set of crown gear teeth 231 are in permanent engagement with the reversing member 207.

The drive control member 204 also comprises a stop feature 232 which is configured to abut a corresponding stop feature 233 of the secondary drive control member 205 shown in FIG. 31 at the end of a dose dispense operation. The stop feature 232 of the drive control member 204 is arranged at an inner face 234 of the drive control member 204 being perpendicular to the second axis 220 and facing towards the dose setting member 203.

FIG. 31 shows the secondary drive control member 205. The secondary drive control member 205 is also mounted on the shaft 221 of the dose setting member 203. The secondary drive control member 205 comprises a through hole 235 wherein the shaft 221 extends through the through hole 235 in the assembled drive assembly 201. The outer face 236 of the secondary drive control member 205 facing away from the dose setting member 203 comprises the stop feature 233 which is configured to abut with the stop feature 232 of the drive control member 204 at the end of a dose dispense operation. An abutment of the stop feature 232 of the drive control member 204 and the stop feature 233 of the secondary drive control member 205 provides a rotational limit to a movement of the drive control member 204 at the end of a dose dispense operation.

Further, the secondary drive control member 205 comprises a perimeter surface 237 which faces away from the second axis in the assembled drive assembly 201. The perimeter surface 237 has a stepped form. In particular, the perimeter surface 237 has an inner area 238 and an outer area 239 wherein the inner area 238 has a slightly smaller diameter than the outer area 239.

On the perimeter surface 237 of the secondary drive control member 205, two sets of gear teeth 240, 241 are arranged. In particular, on the perimeter surface, an inner set of gear teeth 240 and an outer set of gear teeth 241 are arranged. The inner set of gear teeth 240 are arranged on the inner area 238 and the outer set of gear teeth 241 is arranged on the outer area 239.

The inner set of gear teeth 240 is releasably engaged with teeth 242 of the drive control member stop 206 shown in FIG. 26. This engagement causes the secondary drive control member 205 to be rotationally constrained, i.e. the secondary drive control member 205 is prevented from rotating relative to the drive control member stop 206 and thereby from rotating relative to the housing of the drug delivery device when the inner set of gear teeth 240 is engaged with teeth 242 of the drive control member stop 206.

The outer set of gear teeth 241 is configured to engage with the dose setting member 203 during dose dialing.

FIGS. 32 and 33 show perspective views of the dose setting member 203. The dose setting member 203 comprises the shaft 221 defining the second axis 220. In particular, the shaft 221 is integrally formed with the dose setting member 203. The shaft 221 is held in the housing of the drug delivery device such that the dose setting member 203 is prevented from rotating relative to the housing, but can translate axially along the second axis 220. Further, the dose setting member 203 is permitted to rotate relative to the housing if it has previously been moved axially along the second axis 220.

The dose setting member 203 further comprises an indicator 243 arranged at its outer surface facing away from the drive control member 204. On the indicator 243, dial numbers and graduations are printed. In particular, the housing comprises a pointer 252, which is shown in FIG. 38, wherein the pointer 252 points to one of the dial numbers or graduations, thereby indicating the number of a currently set dose. Accordingly, the dose setting member 203 is one of the elements of the drive assembly 201 allowing a user to control the operation of the drive assembly 201. In particular, the dose setting member 204 is used to set the intended dose and the indicator 243 of the dose setting member 204 comprising printed numbers and graduations is used to indicate by alignment with the pointer 252 attached to the housing the currently set dose.

The outer perimeter of the dose setting member 203 is held in the housing of the drug delivery device. In particular, the indicator 243 is held at its perimeter. Further, the axial translation of the dose setting member 203 is limited by features (not shown) on the housing of the drug delivery device and by the secondary drive control member 205.

Further, at an inner surface of the dose setting member 203 facing towards the secondary drive control member 205, gear features 244 are arranged. The gear features 244 of the dose setting member 203 provide a connection with the secondary drive control member 205 when the dose setting member 203 is translated axially to enable dose setting. In particular, the gear features 244 of the dose setting member 203 are configured to engage with the outer set of gear teeth 241 of the secondary drive control member 205.

The inner surface of the dose setting member 203 also acts on the drive control member stop 206 when translated axially during dose setting. In particular, the inner surface of the dose setting member 203 abuts the drive control member stop 206 such that the drive control member stop 206 follows an axial displacement of the dose setting member 203 during dose setting.

The drive control member 204 and the secondary drive control member 205 are located on the shaft 221 integrally formed by the dose setting member 203.

Further, the coupling member 209 is rigidly fixed to an end 245 of the shaft 221. The end 245 of the shaft 221 has a non-circular cross-section rigidly fixing the coupling member 209 to the shaft 221.

The coupling member 209 comprises teeth 264. The teeth 264 may engage the reversing member 207. The reversing member 207 comprises teeth 265 arranged at its outer perimeter. The teeth 264 of the coupling member 209 may engage the teeth 265 of the reversing member 207.

As the coupling member 209 is rigidly fixed to the dose setting member 203, the coupling member 209 follows an axial movement of the dose setting member 203. Depending on the axial position of the dose setting member 203, the teeth 264 of the coupling member 209 are either engaged to the teeth 265 of the reversing member 207 or are arranged at a distance away from the teeth 265 of the reversing member 207. When the teeth 264 of the coupling member 209 are engaged with the teeth 265 of the reversing member 207, a rotation of the coupling member 209 around the second axis 220 results in a rotation of the reversing member 207 around the main axis 219 and vice versa.

The drive control member stop 206 comprises teeth 242, as shown in FIG. 26. Further, the drive control member stop 206 is constrained at its outer surfaces in the housing such that it can only move in a direction that is parallel to the second axis 220. With no user input, the drive control member stop 206 is engaged with the secondary drive control member 205. In particular, the teeth 242 of the drive control member stop 206 are engaged with the outer set of gear teeth 239 of the secondary drive control member 205. Thereby, the secondary drive control member 205 is rotationally fixed to the housing.

An axial movement of the dose setting member 203 causes the drive control member stop 206 to disengage from the secondary drive control member 205, allowing the secondary drive control member 205 to rotate and a new dose end stop to be set.

The actuator 212, shown in FIG. 26, comprises a button 246 that may be pressed by a user. Further, the actuator 212 comprises a shaft 247. The shaft 247 and the button 246 are integrally formed. The shaft 247 extends from the button 246 in the direction parallel to the second axis 220 towards the dose setting member 203. The actuator 212 is constrained by the housing such that the actuator 212 can only move in a direction that is parallel to the second axis 220. Further, splines 230 are arranged at the end of the shaft 247 facing away from the button 246. The splines 230 are engaged with the drive control member 204 when the button 246 is not depressed. This engagement prevents the drive control member from rotating relative to the actuator 212 and thereby from rotating relative to the housing of the drug delivery device. A depression of the button 246 causes the splines 230 to disengage from the drive control member 204. When disengaged from the splines 230, the drive control member 204 is enabled to rotate.

FIG. 34, as well as FIGS. 27 and 28, show the drive assembly 201 in a rest state. The rest state is a state before a dose setting operation is carried out.

The last dose stop drive member 211 comprises a set of gear teeth 248 which are engaged with the secondary drive control member 205. Accordingly, a rotation of the secondary drive control member 205 results in rotating the last dose stop drive member 211 relative to the housing.

Further, the last dose stop drive member 211 comprises a threaded portion 249. The last dose stop member 210 comprises a corresponding thread at its inner surface. The last dose stop member 210 runs on the threaded portion 249 of the last dose stop drive member 211. The last dose stop drive member 211 is constrained to the housing such that it can only rotate relative to the housing, but is prevented from moving axially along a linear axis parallel to the second axis 220 relative to the housing.

The last dose stop member 210 is threadedly engaged with the threaded portion 249 of the last dose stop drive member 211. The last dose stop member 210 is engaged by a spline feature 250 with the housing such that the last dose stop member 210 is prevented from rotating relative to the housing. Moreover, the last dose stop member 210 comprises a stop face. The stop face is configured to engage with the last dose stop drive member 211 when the permitted total number of doses has been selected.

In the rest state, the drive control member stop 206 is engaged with the secondary drive control member 205. Thereby, the secondary drive control member 205 is rotationally locked such that it can not rotate relative to the drive control member stop 206 or the housing of the drug delivery device.

Further, the splines 230 of the actuator 212 are engaged with the drive control member 204. Thereby, the drive control member 204 is rotationally locked such that it can not rotate relative to the actuator 212 and the housing of the drug delivery device. As the drive control member 204 is further engaged to the teeth 225 of the piston rod 214, the piston rod 214 is prevented from moving in a distal direction 215.

The stop feature 232 of the drive control member 204 is in abutment with the stop feature 233 of the secondary drive control member 205.

On the dose setting member 203, the “0” mark is in alignment with the pointer 252 of the housing.

The reversing member 207 is in toothed engagement with the drive control member 204 and the coupling member 209. In particular, the set of crown gear teeth 231 of the drive control member 207 are engaged with the teeth 265 of the reversing member 207. Further, the teeth 265 of the reversing member 207 are engaged with the teeth 264 of the coupling member 209.

As the drive control member 204 is prevented from rotating relative to the housing due to the engagement of the drive control member 204 with the splines 230 of the actuator 212, the coupling member 209 is also prevented from rotating relative to the housing. Thereby, the dose setting member 203 is prevented from rotating relative to the housing, as the coupling member 209 is further rigidly fixed to the end 245 of the shaft 221 of the dose setting member 203.

FIGS. 35 and 36 show the drive assembly 201 in a ready-to-set state. To enable dialing of a new dose, the dose setting member 203 has to be first pushed inwards in a direction along the second axis 220 by the user. Inwards means hereby that the dose setting member 203 is pushed towards the secondary drive control member 205.

When the dose setting member 203 is pushed inwards, this drives the drive control member stop 206 axially along the second axis 220. Thereby, the drive control member stop 206 is disengaged from the secondary drive control member 205. Due to the disengagement from the drive control member stop 206, the secondary drive control member 205 is now allowed to rotate. Simultaneously, the secondary drive control member 205 engages the dose setting member 203 by an engagement of the inner set of gear teeth 240 of the secondary drive control member 205 engaging the gear features 244 of the dose setting member 203.

Moreover, in the ready-to-set state of the drive assembly 201, i.e. when the drive control member 204 has been pushed inwards, the coupling member 209 being rigidly fixed to the drive control member 204 is moved axially along the second axis 220 and is thereby disengaged from the reversing member 207. Due to the disengagement of the coupling member 209 from the reversing member 207, it is prevented that a rotation of the dose setting member 203 results in translating the piston rod 214.

However, as the coupling member 209 is disengaged from the drive control member 204 in the ready-to-set state, the coupling member 209 is now enabled to rotate relative to the housing. Thereby, the dose setting member 203 which is engaged to the coupling member 209 is also enabled to rotate relative to the housing in the ready-to-set state, i.e. after it has been pushed inwards.

Moreover, the drive control member stop 206 follows the axial movement of the dose setting member 203. Accordingly, in the ready-to-set state, the drive control member stop 206 abuts the splined end of the actuator 212, thereby preventing the actuator 212 from being moved axially in a direction towards the dose setting member 203. Accordingly, the actuator 212 cannot be depressed in the ready-to-set state.

Moreover, the drive control member 204 is prevented from rotating relative to the housing due to its engagement with the splines 230 of the actuator 212 in the ready-to-set state.

To set a new dose, a user rotates the dose setting member 203 whilst it is pushed inwards.

FIG. 37 shows the drive assembly 201 in a dose-set state.

Compared to the ready-to-set state shown in FIGS. 35 and 36, the dose setting member 203 has been rotated. As the second drive control member 205 is now engaged to the dose setting member 203, the secondary drive control member 205 follows this rotation.

As there is no spring to compress during the dose setting operation, setting of the dose requires very little torque input.

In this new dose set position, the stop feature 233 of the secondary dose control member 205 has moved to provide a new end stop for the drive control member 204. The secondary drive control member 205 has been relocked in rotation by an engagement with the drive control member stop 206.

FIG. 38 shows a part of the housing 263 comprising a window 251.

As the dose setting member 203 has been rotated, the indicator 243 of the dose setting member 203 has been rotated as well. The set dose is now displayed on the indicator 243 of the dose setting member 203. The set dose can be viewed through the window 251 of the housing. Only a small group of printed numbers is visible through the window 251. A magnifying lens may be arranged in the window 251. Alternatively, the window may comprise a simple cutout in the housing. The pointer 252 on the housing points to the number corresponding to the set dose.

FIG. 39 shows the drive assembly 201 after the dose setting operation has been completed and before the dose dispense operation is initiated.

During dose setting, the drive control member 204 is rotationally fixed relative to the housing by its engagement to the actuator 212. The actuator 212 is configured such that the actuator 212 cannot be depressed while a dose setting operation is carried out. In particular, the drive control member stop 206 abuts the splined end of the shaft 247 of the actuator 212 such that the actuator 212 is prevented from moving in a direction along the second axis 220. Accordingly, a dose cannot be accidently delivered during dose setting as the dose delivery operation has to be initiated by depressing the actuator 212 which is prevented during dose setting.

After the dose setting operation has been completed, the user releases the dose setting member 203. The dose setting member 203 returns via a spring (not shown) to its original outward position, along with the drive control member stop 206. Now, the drive control member stop 206 does not abut the actuator 212 any more such that the actuator is not locked against an axial movement and can now be depressed by a user.

Before the actuator 212 is depressed by a user, i.e. before a dose dispensing operation is initiated, the set dose can be amended, i.e. it can be increased or decreased. To do this, the user has to depress and rotate the dose setting member 203 again.

FIG. 40 shows the initiation of a dose dispensing operation. Further, FIG. 41 shows the drive assembly 201 during a dose dispensing operation.

In order to dispense a dose, the actuator 212 is pressed. This causes the actuator 212 to translate parallel to the second axis 220 and releases the splined connection between the actuator 212 and the drive control member 204. When the drive control member 204 is released, it is driven rotationally. In particular, the spring member 213 exerts a force on the piston rod 214. Specifically, the spring member 213 exerts a force on the first spring seat 261 formed by the bearing 217 of the piston rod 214. As the drive control member 204 is not locked against a rotation, the spring member 213 is enabled to expand. This results in a translation of the piston rod 214 in the distal direction 215. As the teeth 225 of the piston rod 214 are engaged to the inner small diameter pinion gear 227 of the drive control member 204, the drive control member 204 is thereby rotated.

The axial translation of the piston rod 214 allows the bearing 217 to drive the piston 218 forward in a distal direction 215 further into the cartridge 202, thus delivering the dose of the medicinal product.

The drive control member 204 is rotated until its stop feature 232 reaches the new end stop position set by the stop feature 233 of the secondary drive control member 205. The end of the rotation of the drive control member 204 corresponds to the delivery of the dose being finished. When the stop feature 232 reaches the new end stop position, the drive control member 204 is prevented from rotating further relative to the housing. The engagement of the drive control member 204 with the piston rod 214 prevents a further translation of the piston rod 214 in the distal direction, thereby preventing the piston rod 213 from expelling more of the medicinal product from the cartridge 202.

During the dose dispensing operation, the indicator 243 of the dose setting member 203 automatically travels back to its “0” position such that “0′” is displayed in the window 251 of the housing. This is achieved by an interaction of the coupling member 209 and the reversing member 207. During dose dispense, the reversing member 207 is rotated due to its toothed engagement with the drive control member 204.

When the dose setting member 203 is moved outward to its original position after the dose setting has been completed and before the dose dispense is started, the coupling member 209 follows this movement as the coupling member 209 is rigidly fixed to the dose setting member 203. Thereby, the coupling member 209 engages the reversing member 207. Accordingly, the coupling member 209 is coupled via the reversing member 207 to the drive control member 204 during the dose dispense operation. Further, the drive control member 204 is rotated during the dose dispense operation such that this rotation causes the coupling member 209, and hence the indicator 243, to rotate back to its zero display position.

Moreover, the drive assembly 201 comprises a last dose lockout assembly which is shown in FIG. 42. During dose setting, the secondary drive control member 205 rotates and this causes the last dose stop drive member 211 to rotate due to their toothed engagement. This in turn causes the last dose stop member 210, which is prevented from rotating, to translate along the longitudinal axis of the last dose stop drive member 211. During dose dispense, the secondary drive control member 205 does not rotate. Accordingly, the last dose stop drive member 211 also does not rotate.

When the maximum number of doses available has been dialed, the last dose stop member 210 reaches the end of the threaded portion 249 and the stop face of the last dose stop member 210 contacts a similar stop face on the last dose stop drive member 211. This prevents a further rotation of the last dose stop drive member 211. Thereby, also a further rotation of the secondary drive control member 205 and of the dose setting member 203 is prevented such that it is not possible to dial a larger dose. However, the number of units available for the last dose is now shown on the indicator 243 in the normal way before the final units are dispensed. This allows splitting of the dose in two injections if required.

Furthermore, the drive assembly 201 comprises a safety member 253. FIG. 43 shows the drive assembly 201 comprising the safety member 253 in a state in wherein the drive assembly 201 is undamaged. FIG. 44 shows the drive assembly 201 comprising the safety member 253 in a state wherein the drive assembly 201 is damaged.

The safety member 253 is configured to prevent a movement of the piston rod 214 when the drive assembly 201 is damaged. The safety member 253 prevents the spring member 213 from automatically dispensing the remaining contents of the cartridge 202 when the drive assembly 201 is damaged, e.g. when the piston rod 214 is damaged.

The safety member 253 comprises a first safety member part 254 and a second safety member part 255. The first safety member part 254 comprises a strap 256. One end of the strap 256 is fixed to the bearing 217 of the piston rod 214 which corresponds to the first spring seat 261. The strap 256 runs parallel to the piston rod 214. In particular, the strap 256 is arranged to run along the upper main surface 223 of the piston rod 214.

The first safety member part 254 comprises a first engagement member 257 comprising teeth arranged on its surface facing away from the upper main surface 223 of the piston rod 214.

The second safety member part 255 comprises a spring arm 258 which is attached to the housing part 221. The housing part 221 corresponds to the second spring seat 262. The spring arm 258 comprises a second engagement member 259 and a spacer member 260. The spacer member 260 abuts the piston rod 214 with a light spring force. The second engagement member 259 is formed integrally with the spring arm 258. The second engagement member 259 comprises a protrusion which is configured to engage with the teeth of the first engagement member 257 of the first safety member part 254.

The strap 256 of the first safety member part 254 comprising the first engagement member 257 is connected to the first spring seat 261. Further, the second engagement member 259 of the second safety member part 255 is connected to the second spring seat 262. When the first and the second safety member parts 254, 255 are not engaged to each other in the undamaged state of the drive assembly 201, they do not provide a mechanical connection between the first and the second spring seat 262.

When the drive assembly 201 is undamaged, as shown in FIG. 43, the spacer member 260 holds the second engagement member 259 of the second safety member part 255 away from the first safety member part 254 by the tension of the piston rod 214.

Further, FIG. 44 shows a situation wherein the drive assembly 201 is damaged. This damage may result in the piston rod 214 releasing its tension.

For example, when the piston rod 214 breaks or is detached at either end, its tension loosens and the piston rod 214 becomes slack. In this condition, the spacer member 260 is enabled to overcome the now reduced tension of the piston rod 214. Accordingly, the spacer member 260 moves the piston rod 214 in a direction away from the first safety member part 254. This enables the first safety member part 254 to engage with the second safety member part 255. In particular, the teeth of the first engagement member 257 engage with the protrusion of the second engagement member 259.

The engagement of the first and the second safety member parts 254, 255 locks the spring member 213. In particular, the engagement of the first and the second safety member parts 254, 255 fixes the distance between the first and the second spring seat 262 such that the first and the second spring seats 261, 262 are prevented from moving relative to each other, as the first safety member part 254 is fixed to the first spring seat 261 formed by the bearing 217 and the second safety member part 255 is fixed to the second spring seat 262 formed by the housing part 221. When the distance between the spring seats 261, 262 is fixed, the spring member 213 is prevented from relaxing any further.

In particular, the first safety member part 254 is now prevented from moving in the distal direction 215 any further as it is engaged to the housing part 221 via the second safety member part 255. As the first safety member part 254 is fixed to the first spring seat 261 at one end, the first spring seat 261 can not move in the distal direction 215 when the first and the second safety member parts 254, 255 are engaged to each other. This prevents a further movement of the spring member 213 and thereby of the piston rod 214. Accordingly, a further dose dispensing is also prevented.

FIG. 45 shows an exploded view of a drive assembly 301 for a drug delivery device according to a third embodiment. The drive assembly 301 can be operated to deliver variable doses of a medicinal product from a cartridge 302, via a needle (not shown).

The drive assembly 301 is structurally and functionally similar to the drive assembly 201 according to the second embodiment as shown in FIGS. 26 to 44. The main differences of the drive assemblies 201, 301 are the specific embodiments of the spring members 213, 313 and the piston rods 214, 314.

The drive assembly 301 comprises a dose setting member 303, a drive control member 304, a secondary drive control member 305, a drive control member stop 306, a reversing member 307, a reversing member shaft 308, a coupling member 309, a last dose stop 310, a last dose stop drive member 311 and an actuator 312, wherein these parts structurally and functionally correspond to the parts of the drive assembly 201 according to the second embodiment. In particular, the interactions of these parts with each other correspond to the interactions of the corresponding parts of the drive assembly 201 according to the second embodiment.

Furthermore, the drive assembly 301 comprises a spring member 313 and a piston rod 314, which are different from the spring member 213 and the piston rod 214 of the second embodiment. The spring member 313 is configured as a torsion spring. The spring member 313 has two free ends 322, wherein one of which is located in a through hole 323 in the drive control member 304 and the other one in a through hole 324 in the secondary drive control member 305.

The drive assembly 301 comprises a main axis 319. The main axis 319 of the drive assembly 301 corresponds to a longitudinal axis of the cartridge 302. The piston rod 314, extends along the main axis 319 of the drive assembly 301.

The piston rod 314 is configured as a rack and comprises a bearing 317 arranged at the distal end of the piston rod 314. The bearing 317 is adapted to provide a force on a piston 318 arranged in the cartridge 302 in order to expel a medicinal product from the cartridge 302. The piston rod 314 is axially and rotationally constrained in a housing of the drug delivery device so that it can only move in a linear fashion, in particular along the main axis 319 of the drive assembly 301. The position of the piston rod 314 along the main axis 319 is constrained by the drive control member 304. In particular, the teeth 325 of the piston rod are engaged with an inner small diameter pinion gear 327 of the drive control member 304.

Further, the drive assembly 301 defines a second axis 320. The second axis 320 is perpendicular to the main axis 319. In particular, the second axis 320 is defined by a shaft 321 of the dose setting member 303. In the drive assembly 301, the dose setting member 303, the secondary drive control member 305, the drive control member 304 and the coupling member 309 are arranged coaxially on the second axis 320.

The drive assembly 301 is configured to be located in a housing of the drug delivery device. In FIG. 45, the housing is not shown for clarity reasons.

FIG. 46 shows a perspective view of the assembled drive assembly 301 and a cartridge 302 attached to the drive assembly 301.

FIGS. 47 to 58 illustrate the functionality of the drive assembly 301 of the third embodiment, in particular during dose setting and dose dispensing operations. The functionality and the interactions of the different parts of the drive assembly 301 of the third embodiment correspond to the functionality and the interactions of the different parts of the drive assembly 201 of the second embodiment apart from the interactions and structure of the spring member 313 and the piston rod 314. Therefore, the description equally applies to the drive assembly 201 of the second embodiment apart from the details of the spring member 313 and the piston rod 314 and vice versa.

FIG. 47 shows the drive assembly 301 in a rest state. FIG. 48 shows a part of the drive assembly 301 in the rest state.

In particular, the drive control member stop 306 is constrained to a housing of the drug delivery device such that it can only move parallel to the second axis 320. With no user input, teeth 342 of the drive control member stop 306 are engaged with an inner set of gear teeth 340 of the secondary drive control member 305. Thereby, the secondary drive control member 305 is rotationally locked such that it can not rotate relative to the housing of the drug delivery device.

Further, splines 330 of the actuator 312 are engaged with the drive control member 304. Thereby, the drive control member 304 is rotationally locked such that it can not rotate relative to the actuator 312 and the housing of the drug delivery device.

A stop feature 332 of the drive control member 304 is in abutment with a stop feature 333 of the secondary drive control member 305. Thereby, a pre-torque from the spring member 313 is prevented from being applied to the drive control member 322.

FIG. 49 shows a part of the drive assembly in a ready-to-set state. FIG. 50 shows a further part of the drive assembly in a ready-to-set state. FIG. 51 shows the drive assembly in a ready-to-set state.

As can be seen in FIG. 49, the dose setting member 303 has been pushed inwards as indicated by the arrow. Thereby, the drive control member stop 306 has disengaged from the secondary drive control member 305. Thereby, the secondary drive control member 305 is enabled to rotate relative to the housing. Furthermore, the gear features 344 of the dose setting member 303 engage with an outer set of gear teeth 341 of the secondary drive control member 305. Thereby, the secondary drive control member 305 is coupled to the dose setting member 303 such that a rotation of the dose setting member 303 results in a rotation of the secondary drive control member 305.

As can be seen in FIG. 50, when the dose setting member 303 is pushed inwards as indicated by the arrow, the coupling member 309 disengages from the reversing member 307 and, thus from the piston rod 314. Thereby, a translation of the piston rod 314 by a rotation of the dose setting member 303 is prevented.

As can be seen in FIG. 51, in the ready-to-set state an end of the actuator 312 abuts the drive control member stop 306. This abutment prevents an accidental operation of the actuator 312 during dose setting.

When the dose setting member 303 is rotated to set a new dose, the secondary drive control member 305 is rotated, whereby the spring member 313 located in the through hole 324 of the secondary drive control member 305 is wound up.

When the user releases the dose setting member 303, it returns along second axis 320 under a force exerted by a spring (not shown) to its original outward position, along with the drive control member stop 306.

FIG. 52 shows the drive assembly in a dose-set state. FIG. 53 shows a view of an indicator 343 in a dose-set state. FIG. 54 shows a part of a housing 353 in a dose-set state.

As can be seen in FIG. 52, the stop feature 333 of the secondary drive control member has moved, in particular rotated about the second axis 220 during dose setting, to provide a new end stop position for the drive control member 304. Accordingly, the stop feature 332 of the drive control member 304 and the stop feature 333 of the secondary drive control member 305 are out of abutment and located at a defined angular distance from each other. The drive control member 304 has been re-locked in rotation by the actuator 312. Furthermore, the coupling member 309 has been re-engaged to the reversing member 307 and, thus, to the piston rod 314.

As can be seen in FIG. 53, on the indicator 343, dial numbers and graduations are printed. In particular, the indicator 343 is divided into equal segments with the numbers printed along with indicia to mark each increment.

As can be seen in FIG. 54, the housing 353 comprises a pointer 352, wherein the pointer 352 points to one of the dial numbers or graduations, thereby indicating the number of a currently set dose. The set dose may be viewed through a magnifying lens or a simple cut out in the housing 353.

FIG. 55 shows the drive assembly 301 during an initiation of a dose dispensing operation.

In order to dispense a dose, the actuator 312 is depressed as indicated by an arrow. This causes the actuator 312 to translate parallel to the second axis 320 and release the splined connection with the drive control member 304. When released, the drive control member 304 is driven by the spring member 313, for example in a clockwise direction, until its stop feature 332 abuts the stop feature 333 of the secondary drive control member 305. Thereby, the stop features 332, 333 act as a dispense stop. In particular, the stop features 332, 333 limit the travel of the piston rod 314 and of the indicator 343.

The rotation of the drive control member 304 causes the piston rod 314 to move parallel to the main axis 319 and drive the piston 318 in the cartridge 302 forward, thus delivering the dose.

FIG. 56 shows a part of the drive assembly during a dose dispense operation.

During dose dispensing, the dose setting member 303 and, thereby, the indicator 343 travels back to its initial position, i.e. the ‘0’ displayed position. A new dose can be set immediately afterwards if required. The interaction of the coupling member 309 and the reversing member 307 is used to achieve this. During dispense, the reversing member 307 is rotated due to its toothed engagement with the drive control member 304. Due to the toothed engagement of the reversing member 307 and the coupling member 309, this rotation also causes the coupling member 309 and hence the dose setting member 303 to rotate back to its ‘0’ display position.

When the maximum number of doses available has been dialed, the last dose stop 310 reaches the end of the threaded engagement with the last dose stop drive member 311. A stop face on the last dose stop 310 contacts a similar stop face on the last dose stop drive member 311, thereby preventing a setting of a dose larger than an available dose. When the stop faces abut, the last dose stop member is in its end position. The configuration of the last dose stop corresponds to the last dose stop shown in FIGS. 18A to 18C according to the first embodiment. FIG. 18A shows a start position of the last dose stop member and FIG. 18C shows an end position of the last dose stop member.

FIG. 57 shows an alternative embodiment of a piston rod 313 for a drive assembly 301 according to the third embodiment. The piston rod 313 is curved and flexible. However, the piston rod 313 provides a sufficient stability to drive the piston 318 forward during dose dispense.

Due to the curved shape of the piston rod 313, a shorter overall length of the drug delivery device can be achieved.

FIG. 58 shows a drug delivery device 354 according to the alternative embodiment of FIG. 57. The layout of the piston rod 313 allows a curved housing shape offering an improved ergonomic solution for the device 354.

### REFERENCE NUMERALS

101 drug delivery device

102 piston rod

103 first thread

104 second thread

105 first pitch

106 second pitch

107 first inner diameter

108 second inner diameter

109 longitudinal axis of device

111 longitudinal axis of piston rod

111 distal end of device

112 proximal end of device

113 dose setting direction

114 dose cancelling direction

115 axial spline of piston rod

116 housing

117 cartridge holder

118 cartridge

119 piston

120 actuator

121 reset member

122 dose setting member

123 rotation member

124 last dose stop member

125 locking member

126 piston rod nut

127 spring member

128 indicator

129 indication window

130 coupling member

131 cap

132 first engagement feature of actuator

133 second engagement feature of actuator

134 proximal face of piston rod nut

135 flange of locking member

136 spline of locking member

137 spline of piston rod nut

138 bearing of cap

139 snap feature of cap

140 ratchet feature of dose setting member

141 ratchet arm of rotation member

142 housing ratchet feature

143 engagement means of coupling member

144 engagement means of indicator

145 maximum dose abutment of indicator

146 maximum dose abutment of window member

147 window member

148 thread of window member

149 end of dispense stop

150 distal direction

151 proximal direction

152 thread of locking member

153 extended spline of locking member

154 axial groove of rotation member

155 snap feature of actuator

156 engagement feature of coupling member

158 engagement means of window member

159 last dose stop face of last dose stop member

160 last dose stop face of rotation member

161 thread of rotation member

162 engagement features of dose setting member

163 feedback feature

164 arrow

165 arrow

167 protrusions of actuator

168 magnifying element

169 protrusion

170 stop feature of indicator

171 stop feature of cartridge holder

172 opening of actuator

173 indentation

175 constraint features of cap

176 protrusion of last dose stop member

177 axial groove of housing

178 small diameter bearing

179 radial face of ratchet arm

180 drive assembly

181 thread start

182 thread start

183 thread start

184 thread start

185 sloped face of ratchet arm

190 last dose stop drive member

191 main axis

201 drive assembly

202 cartridge

203 dose setting member

204 drive control member

205 secondary drive control member

206 drive control member stop

207 reversing member

208 reversing member shaft

209 coupling member

210 last dose stop member

211 last dose stop drive member

212 actuator

213 spring member

214 piston rod

215 distal direction

216 proximal direction

217 bearing

218 piston

219 main axis

220 second axis

221 shaft

222 main part

223 upper main surface

224 lower main surface

225 teeth of the piston rod

226 through hole

227 inner small diameter pinion gear

228 outer face

229 teeth

230 splines

231 set of crown gear teeth

232 stop feature

233 stop feature

234 inner face

235 through hole

236 outer face

237 perimeter surface

238 inner area

239 outer area

240 inner set of gear teeth

241 outer set of gear teeth

242 teeth of the drive control member stop

243 indicator

244 gear features

245 end of the shaft

246 button

247 shaft

248 set of gear teeth

249 threaded portion

250 spline feature

251 window

252 pointer

253 safety member

254 first safety member part

255 second safety member part

256 strap

257 first engagement member

258 spring arm

259 second engagement member

260 spacer member

261 first spring seat

262 second spring seat

263 housing

264 teeth of the reversing member

265 teeth of the coupling member

301 drive assembly

302 cartridge

303 dose setting member

304 drive control member

305 secondary drive control member

306 drive control member stop

307 reversing member

308 reversing member shaft

309 coupling member

310 last dose stop

311 last dose stop drive member

312 actuator

313 spring member

314 piston rod

315 distal direction

316 proximal direction

317 bearing

318 piston

319 main axis

320 second axis

321 shaft

322 free end of spring member

323 through hole in drive control member

324 through hole in secondary drive control member

325 teeth

326 through hole

327 inner small diameter pinion gear

328 outer face

329 teeth

330 splines

331 set of crown gear teeth

332 stop feature

333 stop feature

334 inner face

335 through hole

336 outer face

337 perimeter surface

338 inner area

339 outer area

340 inner set of gear teeth

341 outer set of gear teeth

342 teeth

343 indicator

344 gear features

345 end of the shaft

346 button

347 shaft

348 set of gear teeth

349 threaded portion

350 spline feature

351 window

352 pointer

353 housing

354 drug delivery device

1. A drive assembly for a drug delivery device, the drive assembly comprising:
a dose setting member for setting a dose of a drug,
an indicator for indicating the size of the set dose,
a piston rod; and
a dispense stop configured to limit at least one of a movement of the piston rod and a movement-of the indicator during dose dispense,
wherein the dispense stop comprises a stop feature configured to move during dose setting.

2. The drive assembly according to claim 1, comprising a spring member for providing a force for dispensing a set dose.

3. The drive assembly according to claim 1, wherein the indicator is configured to move towards an initial position during dose dispensing.

4. The drive assembly according to claim 1, wherein the indicator is configured to be coupled to the piston rod during dose dispensing.

5. The drive assembly according to claim 4, wherein the dose setting member is configured such that an operation of the dose setting member causes a decoupling of the indicator from the piston rod.

6. The drive assembly according to claim 1, wherein the drive assembly comprises an actuator for initiating a dispensing of a dose and wherein the actuator is configured such that an operation of the actuator causes a coupling of the indicator to the piston rod.

7. The drive assembly according to claim 1, comprising a reversing member being permanently coupled to the piston rod, wherein the reversing member is releasably coupled to the indicator.

8. The drive assembly according to claim 1, wherein the stop feature is fixed during dose dispensing.

9. The drive assembly according to claim 1, comprising a further stop feature, wherein the further stop feature is configured such that the movement of the piston rod and/or the indicator is limited by an abutment of the stop feature and the further stop feature.

10. The drive assembly according to claim 9, wherein the further stop feature is configured to move during dose dispensing and to be fixed during dose setting.

11. The drive assembly according to claim 1, wherein the stop feature is configured to rotate during dose setting.

12. The drive assembly according to claim 1, wherein the stop feature is coupled to the indicator at least during dose setting.

13. The drive assembly according to claim 9, wherein the further stop feature is connected to a drive control member, which controls the movement of the piston rod.

14. The drive assembly according to claim 9, wherein the further stop feature is connected to a housing of the drive assembly.

15. The drive assembly according to claim 1, wherein by the movement of the stop feature during dose setting an end stop position of the stop feature is set and wherein the end stop position of the stop feature defines an end stop position of the indicator and/or the piston rod.

16. The drive assembly according to claim 1, wherein the stop feature is connected to a further member, which is coupled to the indicator during dose setting, and wherein the further member is configured to be decoupled from the indicator during dose dispensing.

17. The drive assembly according to claim 1, wherein the drive assembly comprises a last dose stop for preventing a setting of a dose larger than an available amount of the drug, wherein the last dose stop comprises a last dose stop member.

18. A drug delivery device comprising:
a housing; and
a drive assembly at least partially disposed within the housing, the drive assembly comprising:
a dose setting member for setting a dose of a drug,
an indicator for indicating the size of the set dose,
piston rod; and
a dispense stop configured to limit at least one of a movement of the piston rod and a movement of the indicator during dose dispense,
wherein the dispense stop comprises a stop feature configured to move during dose setting.

19. A method comprising:
pushing a dose setting member inwards, the dose setting member disconnecting a drive control member stop from a secondary drive control member; the pushing of the dose setting member enabling the secondary drive control member to rotate and engaging the secondary drive control member with the dose setting member;
rotating the dose setting member, the dose setting member setting a dose to be administered;
releasing the dose setting member, the releasing disconnecting the drive control member stop from an actuator; and
depressing the actuator to cause a spring member to exert a force on a piston rod into a cartridge and administer an injection of a drug.
